(19) United States
(12) Patent Application Publication
(10) Pub. No.: US 2022/0217972 A1
(43) Pub. Date: Jul. 14, 2022

(54) Methods for cryopreservation of sub-millimeter and millimeter scale biological materials

(71) Applicant: Regents of the University of Minnesota, Minneapolis, MN (US)

(72) Inventors: Li Zhan, St. Paul, MN (US); Mingang Li, Maple Grove, MN (US); Thomas Hays, St. Paul, MN (US); John Bischof, St. Paul, MN (US)

(21) Appl. No.: 17/571,802

(22) Filed: Jan. 10, 2022

Related U.S. Application Data
(60) Provisional application No. 63/136,366, filed on Jan. 12, 2021.

(51) Int. Cl. A01N 1/02; A01K 67/033

(52) U.S. Cl. CPC A01N 1/0284; A01K 2227/706; A01K 67/033

(57) ABSTRACT

Methods for cryopreservation of biological samples are provided. The biological samples are sub-millimeter or millimeter scale biological materials. The biological samples are embryos, such as Drosophila embryos. Methods for cryopreservation of Drosophila embryos using cryomesh are provided. The Drosophila embryos are collected, staged and treated to optimize the cryopreservation outcomes upon rewarming. Methods disclosed are efficient for maintaining stocks of Drosophila wild type and mutant strains. Methods are also disclosed for cryopreservation of other terrestrial organism embryos and/or aquatic organism embryos.

### CROSS-REFERENCE TO RELATED APPLICATION

The present application claims priority to and the benefit of U.S. provisional patent application Ser. No. 63/136,366 filed on Jan. 12, 2021, the entire contents of which are incorporated herein by reference.

This invention was made with government support under OD028758-01 awarded by the National Institutes of Health. The government has certain rights in the invention.

### BACKGROUND

Preservation of biological material is valuable in many areas including for medical and biological research. The fruit fly (Drosophila melanogaster), a foundational genetic model organism for biological research in the past century, has driven important discoveries leading to countless biomedical science breakthroughs. There are >160,000 unique genotypes held in individual research laboratories and stock centers worldwide and this number is growing. Currently, the stocks must be manually maintained through frequent and costly transfer of breeding adults to fresh food.

### SUMMARY

In one aspect, the present description relates to a method for cryopreservation of Drosophila embryos. The method includes collecting Drosophila embryos, treating embryos for cryopreservation, wherein the treating includes staging the embryos, dechorionating the embryos, permeabilizing the embryos, loading the embryos with a cryoprotective solution and dehydrating the cryoprotective solution loaded embryos. The method includes transferring the embryos to a cryomesh and cooling the embryos by placing the embryos on the cryomesh in a cryogenic coolant for cryopreservation of the Drosophila embryos. The cryoprotective solution includes a cryoprotective agent (CPA).

The staging may include visually evaluating the gut morphology of the embryo. The staging of the embryos may include incubating the embryos until the embryos are at a stage when head involution and dorsal closure has been completed. The staging of the embryos may include incubating the embryos in an incubator at about 20° C. for about 22 hours.

The dechorionation may include incubating the embryos in about 50 weight percent bleach. The permeabilizing of the embryos may include incubating the embryos in a permeabilization solution. The permeabilizing solution may include D-limonene and heptane. The permeabilization solution may include D-limonene and heptane at about 4:1 volume/volume. The cryoprotective solution may include ethylene glycol (EG), propylene glycol (PG), dimethyl sulfoxide (DMSO) and combinations thereof. The loading of the embryos with the cryoprotective solution may include incubating the embryos in the cryoprotective loading solution. The cryoprotective loading solution may include between about 10 weight percent and about 15 weight percent of ethylene glycol. The dehydrating of the embryos may include incubation in a dehydrating solution. The dehydrating solution may include the CPA and a sugar. The dehydrating solution may include ethylene glycol and sorbitol. The method may further include wicking the cryomesh with the embryos to remove liquid surrounding the embryos prior to placement in the cryogenic coolant.

The method may further include rewarming the embryos after cryopreservation. The rewarming may include rewarming in a rewarming buffer, unloading the CPA from the cryopreserved embryos and culturing the embryos in a medium. The rewarming buffer may include sucrose, trehalose and combinations thereof. The unloading of the CPA may include incubating in a CPA unloading buffer. The CPA unloading buffer may include sucrose. The culturing may include culturing the embryos in Schneider's medium for between about 8 hours and about 24 hours to form larvae. The method may further include allowing the larvae to hatch and form adult Drosophila. The Drosophila may include a wild-type strain or a mutant strain. The Drosophila may include a mutant strain with a mutation and wherein the mutant strain is genetically modified while maintaining the mutation to improve the survival rates after cryopreservation.

In another aspect, the present description relates to a method for maintaining stocks of Drosophila strains. The method includes collecting Drosophila embryos, treating embryos for cryopreservation, wherein the treating includes staging the embryos, dechorionating the embryos, permeabilizing the embryos, loading the embryos with a cryoprotective solution and dehydrating the cryoprotective solution loaded embryos, transferring the embryos to a cryomesh and cooling the embryos by placing the embryos on the cryomesh in a cryogenic coolant for cryopreservation of the Drosophila embryos and rewarming the embryos after cryopreservation and culturing the rewarmed embryos in medium. The cryoprotective solution includes a cryoprotective agent (CPA). The method may minimize the genetic drift in stocks. The method may halt introduction of further mutations due to genetic drift. The method may stabilize the strain genotypes during stock maintenance. The staging may include visually evaluating the gut morphology of the embryo.

The staging of the embryos may include incubating the embryos until the embryos are at a stage when head involution and dorsal closure has been completed. The staging of the embryos may include incubating the embryos in an incubator at about 20° C. for about 22 hours.

The dechorionation may include incubating the embryos in about 50 weight percent bleach. The permeabilizing of the embryos may include incubating the embryos in a permeabilization solution. The permeabilizing solution may be D-limonene and heptane. The permeabilization solution may be D-limonene and heptane at about 4:1 volume/volume. The cryoprotective solution may include ethylene glycol (EG), propylene glycol (PG), dimethyl sulfoxide (DMSO) and combinations thereof. The loading of the embryos with the cryoprotective solution may include incubating the embryos in the cryoprotective loading solution. The cryoprotective loading solution may include between about 10 weight percent and about 15 weight percent of ethylene glycol. The dehydrating of the embryos may include incubation in a dehydrating solution. The dehydrating solution may include the CPA and a sugar. The dehydrating solution may include ethylene glycol and sorbitol. The method may further include wicking the cryomesh with the embryos to remove liquid surrounding the embryos prior to placement in the cryogenic coolant.

In yet another aspect, the present description relates to a method for cryopreservation of embryos. The method includes collecting embryos, treating embryos for cryopreservation, wherein the treating includes staging the embryos, dechorionating the embryos, permeabilizing the embryos, loading the embryos with a cryoprotective solution and dehydrating the cryoprotective solution loaded embryos, transferring the embryos to a cryomesh and cooling the embryos by placing the embryos on the cryomesh in a cryogenic coolant for cryopreservation of the embryos. The embryos may include of Drosophila embryos. The cryoprotective solution includes a cryoprotective agent (CPA). The permeabilizing of the embryos includes incubating the embryos in a permeabilization solution. The permeabilizing solution may be D-limonene and heptane. The permeabilization solution may be D-limonene and heptane at about 4:1 volume/volume. The cryoprotective solution may include ethylene glycol (EG), propylene glycol (PG), dimethyl sulfoxide (DMSO) and combinations thereof. The loading of the embryos with the cryoprotective solution may include incubating the embryos in the cryoprotective loading solution. The cryoprotective loading solution may include between about 10 weight percent and about 15 weight percent of ethylene glycol. The dehydrating of the embryos may include incubation in a dehydrating solution. The dehydrating solution may include the CPA and a sugar. The dehydrating solution may include ethylene glycol and sorbitol. The method may further include wicking the cryomesh with the embryos to remove liquid surrounding the embryos prior to placement in the cryogenic coolant.

In the following detailed description of illustrative examples, reference is made to specific embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made.

Features or limitations of various embodiments described herein, however important to the examples in which they are incorporated, do not limit other embodiments, and any reference to the elements, operation, and application of the examples serve only to define these illustrative examples. Features or elements shown in various examples described herein can be combined in ways other than shown in the examples, and any such combinations is explicitly contemplated to be within the scope of the examples presented here. The following detailed description does not, therefore, limit the scope of what is claimed.

All patents, publications or other documents mentioned herein are incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a cryopreservation method using cryomesh.

FIG. 2A is a schematic overview of cryopreservation procedures for Drosophila melanogaster embryos and detailed pictorial illustration for critical steps.

FIG. 2B are images of embryo gut morphology under dissecting and compound microscopes after different incubation times at 20° C.

FIG. 2C are images of embryos at different steps during embryo cryopreservation.

FIGS. 3A-3O are plots of cryopreservation protocol optimization using strain, dM2. Box and horizontal line represent standard deviation and mean respectively, whiskers represent max and min. Each data point represents single experiment using >300 embryos, n≥3. Multivariate analysis of variance (MANOVA) and Tukey's post hoc were used for statistical analysis. ns, p>0.05; * p≤0.05; ** p≤0.01; *** p≤0.001; **** p≤0.0001.)

FIG. 3A is a plot of post-cryopreservation survival using embryos of different age.

FIG. 3B is a plot of embryo survival after different soaking time in the LH solution (i.e., permeabilization solution).

FIGS. 3C-3D are plots of post-dehydration and cryopreservation survival using different dehydration time in 39 weight percent EG+9 weight percent sorbitol.

FIGS. 3E-3F are plots of post-dehydration and cryopreservation survival in different dehydration CPAs.

FIG. 3G is a plot of post-cryopreservation survival using different sugars in dehydration CPA.

FIG. 3H is a plot of post-cryopreservation survival with or without liquid remaining on the cryomesh before vitrification.

FIGS. 3I-3J are plots of post-dehydration and cryopreservation survival using different CPAs and cocktails.

FIGS. 3K-3L are plots of post-cryopreservation survival using different CPA unloading methods (FIG. 3K) and different embryo culture methods (FIG. 3L). (In FIGS. 3A-3L optimal conditions were labelled in red.)

FIG. 3M is a plot of survival after each step of the cryopreservation process.

FIG. 3N is a plot of results from two volunteers who were trained to perform the cryopreservation.

FIG. 3O is a plot of post-cryopreservation survival after different storage time in liquid nitrogen.

FIG. 4A is an image of a thermocouple for cooling and warming rate measurement. (1) thermocouple alone on the cryomesh and (2) thermocouple in contact with dehydrated embryos on the cryomesh. Red arrows indicate the thermocouple junction.

FIG. 4B is a plot of measured cooling and warming rate using liquid nitrogen and slush nitrogen in two settings described in (FIG. 4A).

FIG. 4C is a plot of post cryopreservation survival using liquid nitrogen and slush nitrogen.

FIG. 4D is a schematic of the geometry of dehydrated embryos on the cryomesh for the modeling of warming rates. Embryo 1 represents minimal contact with the cryomesh, Embryo 2 represents maximal contact with the cryomesh.

FIG. 4E is an image of warming rates at different cross sections through the embryo center point.

FIG. 4F is a flow chart for evaluation of male to female ratio, fertility and lethality post cryopreservation across multiple generations.

FIG. 4G is an image of PCR analysis that confirmed the original mutation in M2 was maintained after cryopreservation of multiple generations and different storage time in liquid nitrogen.

FIG. 4H is a table of post cryopreservation evaluation after multiple generations and different storage time in liquid nitrogen.

FIGS. 5A and 5B are schematics diagrams of prior art tools/devices for embryo permeabilization (FIG. 5A) and slush nitrogen preparation (FIG. 5B) for vitrification.

FIG. 5C is an image of a simple nylon mesh basket used for embryo permeabilization.

FIG. 5D is an image of a cryomesh used for vitrification. Scale bar is 1 cm.

FIG. 6 is a plot of temperature recording inside the incubator vs. room environment (i.e., lab). The incubator temperature was set to 20° C. to provide robust control of the embryo age for cryopreservation. Fluctuation of the room temperature will lead to inconsistent embryo age therefore inconsistent cryopreservation outcomes as the embryo developmental rate is temperature sensitive.

FIGS. 7A-7C are plots of the age of the flies (strain M2) used for embryo collection impacts cryopreservation outcome. FIG. 7A is a plot of the embryo hatch frequency using 1-4 day old flies for embryo collection. FIG. 7B is a plot of embryo hatch frequency using 9-12 day old flies for embryo collection. FIG. 7C is a plot of comparison of post cryopreservation survival using flies of different ages for embryo collection. p value for hatch rate is 0.651, for adult rate is 0.018. Box and horizontal line represent standard deviation and mean respectively, whiskers represent max and min. Red boxes present embryo hatch rate (i.e., embryo to larvae) and blue boxes represent adult rate (i.e., resulting larvae to adults).

FIGS. 8A-8B are electron microscope (EM) images of WC¹¹¹⁸ embryos before and after permeabilization. FIG. 8A is before permeabilization, a wax layer can be identified outside the vitelline membrane (VM). White and red dashed lines indicate the boundaries of wax layer and VM, respectively. FIG. 8B is after permeabilization, wax layer was removed. Scale bar is 200 nm.

FIG. 9A is a schematic of showing removal of the CPA from cryomesh prior to vitrification.

FIGS. 9B-9C are plots of weight change and cooling/warming rates before vs. after removing the CPA on the cryomesh. FIG. 9B is plot of weight change of the cryomesh (Dm) with CPA removed (i.e., the weight of dehydrated embryos) vs. CPA remaining (the weight of dehydrated embryos+CPA solution). FIG. 9C is a plot of cooling and warming rates measured by a thermocouple with CPA removed vs. CPA remaining on the cryomesh. Removing the CPA greatly improved both the cooling and warming rates. The 532 embryos were used.

FIGS. 10A-10C are images of embryos during cryopreservation. FIG. 10A is an image of dehydrated embryos on the cryomesh after removing CPA solution. FIG. 10B is an image of liquid exchange across the vitelline membrane during CPA unloading. Floating embryos show tiny liquid droplets leaving the embryo surface. FIG. 10C is an image of dehydrated embryos in liquid nitrogen. Embryos circled in black are vitrified embryos showing transparent appearance. Red arrow indicates a crystallized embryo (i.e., failure). Scale bar is 500 μm.

FIGS. 11A-11B show simulated temperature profile of embryos and nylon mesh during rewarming. FIG. 11A is a schematic diagram of embryos 1 and 2, the 4 points selected for evaluation during modeling including the center point of embryo 1, the center point of embryo 2, the center point of nylon between embryo 1 and embryo 2, and the center point of nylon far away from embryos. FIG. 11B is a plot of simulated temperature profile at the 4 points from FIG. 11A. As the Nylon (mesh 1 and mesh 2) rewarmed faster they are able to diffuse heat towards the embryos thereby enhancing their warming rates.

FIGS. 12A-12D (S8) show simulated warming rate of embryos surrounded by CPA solutions. FIG. 12A is a schematic diagram of a protocol using polycarbonate filter paper to carry the embryos and CPA solutions. Two embryos were included in the model, labelled as “1” and “2”. FIG. 12B is a schematic diagram of a protocol using a copper grid to carry the embryos and CPA solutions. The two embryos were included in the model, labelled as “1” and “2”. FIGS. 12 C-12D are images of simulated warming rates of embryos using the methods of FIGS. 12A-12B. Note that these rates are an order of magnitude slower than rates for embryos without the CPA solutions.(FIG. 4E)

FIG. 13 is a plot of post cryopreservation survival comparison using cryobuffer vs. Schneider medium to prepare CPA solutions and unloading solutions. No significant (ns) difference was observed between these cases. p value for hatch rate is 0.704, for adult rate is 0.86. The use of cryobuffer will greatly reduce the cost of cryopreservation.

FIGS. 14A-14C are plots of the effect of embryo age on normalized post-cryopreservation survival using various strains, strain WC1 (FIG. 14A), WC3b (FIG. 14B) and S7 (FIG. 14C).

FIGS. 15A-15B are plots of the effect of soaking time in the permeabilization solution (LH solution) on normalized survival using various strains, strain WC (FIG. 15A), M2-3b (FIG. 15B).

FIGS. 16A-16B are plots of the effect of unloading methods on normalized post cryopreservation survival using various strains, strain NS1 (FIG. 16A), WC-1b (FIG. 16B).

FIGS. 17A-17B are plots of the effect of cryogen on normalized post cryopreservation survival using various strains, strain WC (FIG. 17A), yw1 (FIG. 17B).

FIGS. 18A-18B are plots of the effect of embryo culture methods on normalized post cryopreservation survival using various strains, strain GFP (FIG. 18A), M2-3b (FIG. 18B).

FIGS. 19A-19C are plots of the effect of dehydration time on normalized post cryopreservation survival using various strains, strain WC3b (FIG. 19A), WC (FIG. 19B), WC3 (FIG. 19C). 39 weight percent EG+9 weight percent sorbitol was used.

FIGS. 20A-20B are plots of the effect of dehydration CPA on normalized post cryopreservation survival using various strains, strain GFP (FIG. 20A), WC (FIG. 20B). 9 min dehydration time was used.

FIGS. 21A-21B are plots of the effect of permeable CPA (or cocktail) on normalized post cryopreservation survival using various strains, strain S7 (FIG. 21A), WC (FIG. 21B). 13 weight percent CPA (or cocktail) was used for first step loading, 39 weight percent CPA (or cocktail) +9 weight percent sorbitol was used for dehydration. 9 min dehydration time was used.

FIGS. 22A-22B are plots of the age of the flies used for embryo collection impacts cryopreservation outcome using various strains; strain WC (FIG. 22A, strain WC2 (FIG. 22B).

FIG. 23 shows plots of the hatch frequency of embryos incubated at 24° C. 1 hour embryo collection from various strains were tested. Some strains (i.e., M2, WC, GFP) have a narrow distribution of embryo hatch time, indicating that embryo stage uniformity is high therefore potential higher post cryopreservation survival. Some strains (i.e., S1, NS1) have a broad distribution of embryo hatch time. Some strain hatched earlier (i.e., S7)

FIG. 24A is a diagram of the crossing scheme for NS1.

FIGS. 24B-24E are plots of stepwise survival of S1, NS1 and GFP during cryopreservation. FIG. 24B is a plot of normalized post-permeabilization survival of S1, GFP and NS1. FIG. 24C is a plot of normalized post 13 weight percent EG treatment survival of S1, GFP and NS1. FIG. 24D is a plot of normalized post-dehydration survival of S1, GFP and NS1. FIG. 24E is a plot of normalized post-cryopreservation survival of S1, GFP and NS1.

FIGS. 25A-25B is a suggested flowchart for testing the cryopreservation protocol in Drosophila labs and stock centers. FIG. 25A is a flowchart of a practice run of the protocol using one of the high survival strains. This step is optional but will provide a good benchmark. FIG. 25B is a flowchart of adoption of the protocol described herein for new strains in other labs.

FIGS. 26A-26B are images of examples of wet mesh and dry mesh after a dip step in isopropanol. FIG. 26A is an image of a wet mesh that can be identified by cloudiness visible to the naked eye indicating liquid at the bottom of the mesh basket. FIG. 26B is an image of a dry mesh that is recognized due to increased transparency as evidenced by the ability to see through the mesh. The arrows indicate embryos. Scale bar is 1 cm.

### DEFINITIONS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The terms “and/or” means one or all of the listed elements or a combination of any two or more of the listed elements.

The terms “comprises,” “comprising,” and variations thereof are to be construed as open ended—i.e., additional elements or steps are optional and may or may not be present.

Unless otherwise specified, temperatures referred to herein are based on atmospheric pressure (i.e. one atmosphere).

“Cryopreservation” as referred to herein relates to preservation of a biological sample/specimen at cryogenic temperatures. Cryopreservation includes cooling/freezing the biological sample below subzero temperatures in order to shut down metabolic/chemical activity which can provide long term storage of biomaterials. Cryopreservation of a biological sample may also include warming the biological sample to recover the function/activity of the biological sample.

“Cryogenic” or “Cryogenic temperature” as referred to herein relates to a temperature below sub-zero. Cryogenic temperature can be from −80° C. (112° F.) to absolute zero (−273° C. or −460° F.).

“Cryogenic coolant” as referred to herein relates to a substance that is at a cryogenic temperature, e.g. liquid nitrogen, slush nitrogen.

“Cryoprotective solution” as used herein relates to a solution that includes one or more cryoprotective agent(s) (CPA(s)). Cryoprotective solution may be referred to as “CPA solution” or “CPA”. “Cryoprotective solution”, “CPA solution”, and “CPA” are used interchangeably herein.

“Cryobuffer” as referred to herein relates to an isotonic buffer that is used as the carrier solution for CPA and unloading solution to cryopreserve the Drosophila embryos.

“Cryotool” as referred to herein relates to a cryoresistant tool that can handle a biological sample. The cryotool can, for example, remove a sample from a cryogenic environment. The biological sample may also rest or reside in the cryoscoop during a warming protocol.

“Cryomesh” as referred to herein relates to a cryoresistant tool that can handle a biological sample. The cryomesh can, for example, retain a biological sample on the filaments of the mesh while enabling the removal of any cryoprotective solution surrounding the biological sample.

“Vitrification” as referred to herein relates to a biological sample that has attained a glassy, amorphous structure when cryopreserved. Vitrified samples have less 0.1% V/V of ice crystallization in the sample.

“Crystallized” sample as referred to herein relates to a biological sample that has attained some crystalline structure and may not produce a viable biological sample upon warming to room or physiological temperature. Crystallized samples may also be referred to herein as unvitrified samples, non-vitrified samples, or devitrified samples. These terms are used interchangeably herein.

“High-throughput” as referred to herein relates to the use of automation of a system or other methods to rapidly process a large number of samples in short amount of time.

“Biological specimens” or “biological samples” or “biological material” are used interchangeably and as referred to herein relate to cells, germplasm, cell aggregates, embryos, oocytes and the like. The germplasm can be from a variety of species including, for example, coral germplasm, mammalian germplasm, invertebrate germplasm and the like. The biological samples can be unicellular organisms such as bacteria, protozoa and the like. The embryos and oocytes can be, for example, from invertebrates such as Drosophila, mosquito and others, and vertebrates such as fish, amphibians, mammals, humans and others. The biological samples can be related to commercially relevant or endangered species (i.e. agriculture, aquaculture and biodiversity).

The term “embryos” as referred to herein relates to biological material of a multicellular organism in an early stage of development. Embryos are formed after fertilization in organisms that reproduce sexually. Embryos as used herein can include those from terrestrial and aquatic organisms. Embryos include, for example, insect embryos, fish embryos, amphibian embryos, plant embryos and the like.

Biological samples can include other components to aid in the cryopreservation process, e.g. cryopreserving agent, buffer or other media that are present when the biological sample is prepared, transferred and/or cryopreserved. The size of the biological sample may be characterized by the longest dimension of the biological sample or specimen.

The term “Drosophila” as referred to herein relates to the genus Drosophila and all the species within this genus including Drosophila melanogaster, a fruit fly. It will be understood that Drosophila can include all species of Drosophila and all are within the scope of this description. “Drosophila”, “fruit fly” and “Drosophila melanogaster” will be used synonymously and interchangeably herein.

The term “sub-millimeter” sample as referred to herein relates to a biological sample that is equal to or less than about a millimeter.

The term “millimeter” sample as referred to herein relates to a biological sample that is equal to or more than about a millimeter.

The term “dechorionation” as referred to herein relates to a treatment of embryos that removes completely the outer case/membrane, named chorion, of the embryos.

The term “permeabilization” as referred to herein relates to a treatment that allows a substance such as CPA to enter the interior of specimen, e.g. embryo.

### DETAILED DESCRIPTION OF ILLUSTRATIVE EMBODIMENTS

The present description is directed to systems and methods for cryopreservation of biological materials and rewarming of the cryopreserved biological materials. The present description includes methods for cryopreservation of sub-millimeter and/or millimeter scale biological materials. The present description can include a cryopreservation system that includes the use of a cryomesh in the cryopreservation protocols. The cryomesh can enable the retention of the biological material on the surface of the mesh and removal of cryoprotective agent surrounding the biological material prior to cryopreservation. Methods described herein include methods for cryopreserving the biomaterials with minimal to no cryoprotective agent solution surrounding the sample. Methods include rewarming the cryopreserved sample that is viable for the desired end use. In one embodiment, the biomaterials that are cryopreserved using the methods described herein are embryos. In one embodiment, embryos of Drosophila melanogaster are cryopreserved and rewarmed using the cryomesh in the methods described herein. The rewarmed embryos can mature into adult fruit flies.

High-throughput cryopreservation of biological material, for example, embryos, can be performed using the systems and methods described herein. Well-established, reproducible cryopreservation of biological material can provide a unique opportunity to preserve and expand the use of important biological material.

Cryopreservation can allow viable cells and tissues to be preserved over time in the hypothermic, frozen, or vitrified (glassy) state. This disclosure describes systems, compositions and methods that may be used to cool biological samples and rewarm cryopreserved biological samples from cryogenic temperatures. The systems, methods and compositions described herein are useful in, for example, cooling sub-millimeter—or millimeter-scale cryopreserved biological samples such as, for example, Drosophila embryos and the like. The cryopreservation systems described herein advantageously can be used in methods to process biological samples for long-term storage by cryopreservation and also rewarming of the cryopreserved material. High-throughput techniques can be adapted for processing a large number of samples during cryopreservation and rewarming.

The systems and methods described herein can preserve and restore the integrity of the biological samples upon rewarming. The cooling of the biological sample can result in vitrification of the sample. In one embodiment, this description is directed to systems and methods that can include cooling that can achieve sufficiently high cooling rates to exceed the critical cooling rates (CCRs) of the CPAs to produce adult fruit flies post-cryopreservation from cryopreserved embryos.

In some embodiments, the present description can include a cryopreservation system. The cryopreservation system can include a cryomesh tool for the cryopreservation of a biological sample. In some embodiments, cryomesh can include a handle and a mesh attached to the handle. Advantageously, the cryomesh is a simple, versatile platform that can be used for high throughput cryopreservation (cooling and rewarming) of biological samples, e.g. biological samples in the sub-millimeter or millimeter range, and which can provide capability for rapidly increased cooling and rewarming rates over currently applied approaches.

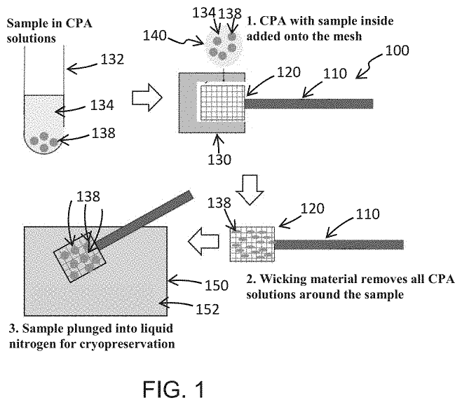

FIG. 1 shows one embodiment of a cryopreservation system and method that includes cryomesh 100. Cryomesh 100 can include handle 110 and mesh 120. Handle 110 can be made from a variety of materials. The handle material can be cryoresistant. The material may be rigid. The material may be sufficiently rigid to hold the mesh in place when the biological sample is placed on the mesh. Handle 110 may be made from, for example, plastics, wood, metal and the like. Handle 110 can include plastics such as acrylics, polyesters, silicones, polyurethane, halogenated plastics, polyethylene, polypropylene, polystyrene, polyvinyl chloride and the like. In one embodiment, handle 110 is made of a plastic.

The length of handle 110 can vary and can be dependent on the specific need and the desired use. Any length of handle 110 may be used for cryomesh 100. In some embodiments, the length of handle 110 can be between about one inch and about 48 inches. In some embodiments, the length of handle 110 can be between about 6 inches and 36 inches; or between about 12 inches and about 24 inches. Handles outside of these ranges are also within the scope of this description.

Cryomesh 100 can be assembled by purchasing the handle, for example, from Thermo Fisher Scientific in Waltham, Mass. and purchasing the mesh, e.g. nylon mesh, for example, from Amazon.com in Seattle, Wash.

Mesh 120 can be permanently and/or removably attached to handle 110 of cryomesh 100. As shown in FIG. 1, mesh 120 can be attached to handle 110 in a manner that mesh 120 can retain biological sample 138 on mesh 120 when cryoprotective agent (CPA) solution 134 surrounding sample 138 is removed. Mesh 120 is a porous mesh. The gaps within mesh 120 are sized such that the biological specimen placed on mesh 120 will not pass through the gaps but will be retained on mesh 120. CPA solution 134 can be substantially removed or wicked away from biological sample 138 by a variety of methods. In one embodiment, CPA solution 134 is removed from sample 138 by wicking solution 134 by wicking material 130. Wicking material 130 can be, for example, wicking paper. In one embodiment, CPA solution 134 may also be removed by the use of an external vacuum.

The characteristics of mesh 120 can vary and can be selected depending on the desired use of cryomesh 100 and biological sample 138. In one embodiment, mesh 120, for example, can vary depending on the size and nature of the biological sample. The characteristics of mesh 120 can impact the ability of biological sample 138 to adhere to and/or be retained on mesh 120. The characteristics of mesh 120 can affect the density of specimen that can be packed onto mesh 120. The characteristics of mesh 120 can affect the ability to wick away excess CPA solution 134.

The characteristics of mesh 120 can vary depending on the materials, mesh patterns, mesh density, mesh filament geometry, mesh filament surface and the like. Materials for mesh 120 can include, for example, plastics, metals, nylon, carbon elastomer and the like. Plastics can include, for example, acrylics, polyesters, silicones, polyurethane, halogenated plastics, polyethylene, polypropylene, polystyrene, polyvinyl chloride, graphite, polydimethylsiloxane and the like. Mesh may include other natural and manmade polymers and all are within the scope of this description. Mesh may also include metals such as, for example, aluminum, copper, stainless steel and the like.

Mesh 20 can include a variety of sizes for the openings between the filaments within mesh 20. The size of the openings can vary and can be dependent on the size of the biological sample that is cryopreserved. In one embodiment, the size of the openings is less than about one millimeter; or less than about 750 micrometers; or less than about 500 micrometers; or less than about 250 micrometers; or less than about 100 micrometers; or less than about 50 micrometers; or less than about 10 micrometers.

In some embodiments, the openings in the mesh can be greater than about one micrometer; or greater than about 50 micrometers; or greater than about 100 micrometers; or greater than about 250 micrometers; or greater than about 500 micrometers; or greater than about 750 micrometers; or greater than about 900 micrometer.

Patterns for mesh 120 can include, for example, plain weave, twill weave, dutch weave and the like. The density of mesh 120 can include, for example, a range from about 50 to about 1250 mesh per inch. In some embodiments, the density of mesh 120 can be between about 100 mesh per inch and about 1000 mesh per inch; or between about 250 mesh per inch and about 500 mesh per inch. The filament geometry of mesh 120 can include, for example, cylindrical, rectangular and the like. In some embodiments, mesh filament surfaces can include, for example, hydrophilic surfaces. In some embodiments, mesh filament surfaces can include, for example, hydrophobic surfaces.

In some embodiments, the mesh size can impact the total amount of biological specimen that can be cryopreserved. In some embodiments, the length of the mesh can be between about 1 cm and about 30 cm; or between about 5 cm and about 20 cm; or between about 10 cm and about 15 cm. Other lengths outside of this range are also within the scope of this description.

In some embodiments, the width of the mesh can be between about 1 cm and about 30 cm; or between about 5 cm and about 20 cm; or between about 10 cm and about 15 cm. Other widths outside of this range are also within the scope of this description.

In some embodiments, the thickness of the mesh can be between about 0.05 and about 0.1 mm; or between about 0.1 and about 0.3 mm; or between about 0.3 and about 0.5 mm. Other thicknesses outside of this range are also within the scope of this description.

The mesh can be in a variety of shapes and all are within the scope of this description. In some embodiments, the mesh is in the shape of a square, a rectangle, a circle and the like.

In some embodiments, cryomesh 100 can be incorporated into an automated or “assembly-line” type approach (e.g. a continuous length or coiled cryomesh).

In some embodiments, the characteristics of mesh 120, e.g. mesh pattern, mesh density, filament geometry (e.g. shape, size), material and the like, can impact the cooling rates experienced by the loaded biological specimen under convective cooling. The cooling rate, for example, can be impacted through contact area and heat transfer characteristics of mesh 120.

In some embodiments, the material and geometry of the mesh can be designed for low thermal mass (mass of the mesh*heat capacity of the mesh material) and high thermal conductivity. The contact area between the biomaterial and the mesh can be increased. Those combined conditions can lead to desired faster cooling/warming rate.

In some embodiments, the characteristics of mesh 120, e.g. mesh pattern, mesh density, filament geometry (e.g. shape, size), material and the like, can impact the rewarming experienced by the loaded biological specimen under convective rewarming. The rewarming rate, for example, can be impacted through contact area and heat transfer characteristics of mesh 120.

Without being bound by any theory, the desired success across a range of biological specimen may require optimization of the cryomesh design parameters to achieve the required loading, cooling, and rewarming rates for specific applications.

In some embodiments, the use of cryomesh in the cryopreservation methods can increase the cooling and rewarming rates and/or increase the throughput over prior art methods. In some embodiments, the cooling rates can be greater than about 25,000° C./min; or greater than about 30,000° C./min; or greater than about greater than about 40,000° C./min; or greater than about 50,000° C./min; or greater than about 60,000° C./min; or greater than about 70,000° C./min; or greater than about 80,000° C./min.

In some embodiments, the warming rates can be greater than about 100,000° C./min; or greater than about 150,000° C./min; or greater than about greater than about 200,000° C./min; or greater than about 300,000° C./min; or greater than about 400,000° C./min; or greater than about 500,000° C./min.

The present description can further include methods that use the cryomesh described herein in methods for cryopreservation of biological samples. The method can include the use of a cryomesh for vitrification and rewarming of the biological specimen. The method can maintain high cooling and/or rewarming rates. In some embodiments, the cryopreservation method can include cooling the biomaterial specimen. The method can include transferring the biomaterials in a CPA solution to the mesh of a cryomesh. The biomaterials in the CPA solution can be transferred onto the mesh in a variety of methods. In some embodiments, a volume of CPA solution with the biological specimen may be placed on the mesh of the cryomesh. The placement of the biomaterials and the CPA solution onto the mesh can result in some or most of the CPA solution being removed from the biomaterials by drainage of the CPA solution through the openings in the mesh. In some embodiments, a wicking material and/or an external vacuum can be used to remove or wick away the CPA solution around the biological sample. The cryomesh with the biological specimen can then be submerged into a cryogenic coolant to rapidly cool the specimen. Advantageously, wicking the CPA solution around the biological sample can reduce the toxicity of the CPA to the biological specimen during cryopreservation.

In one exemplary embodiment, as shown in FIG. 1, biological specimen 138 is combined with CPA solution 134 in vessel 132. In some embodiments, the method can include combining the biomaterials with a CPA solution in vessel, e.g. a test tube, a pan and the like. The biomaterials, the CPA solutions are described in more detail below. A volume of droplet 140 that includes CPA solution 134 and specimen 138 is transferred onto mesh 120 of cryomesh 100. Some of CPA solution 134 drains through gaps of mesh 120. In some embodiments, wicking material 130 may be placed adjacent to mesh 120 to wick CPA solution 134 away from biological specimen 138. It is advantageous to remove all or most of the CPA solution 134 from being in contact with biological specimen 138 prior to cooling. In some embodiments, an external vacuum may also be used to remove all or most of the CPA solution 134 from biological specimen 138.

In some embodiments, the wicking can remove all of the CPA solution around the biological sample; or greater than about 90% of the CPA solution; or greater than about 80% of the CPA solution; or greater than about 50% of the CPA solution around biological sample.

In some embodiments, the wicking material may be fibrous. In some embodiments, the wicking material may be placed on, placed below and/or be resting on/around the mesh to advantageously wick any moisture that may be present in the sample. The fibrous wicking material can be, for example, a fibrous tissue. The thickness of the fibrous wicking material can vary and is within the thickness such that the surface receiving the biological sample can be maintained at a cryogenic temperature. The fibrous wicking material can have a thickness of at least about 0.1 mm. In some embodiments, the thickness of the fibrous wicking material is between about 0.1 mm and about 2 mm. Thickness outside of this range are also within the scope of this disclosure.

The method can further include placing mesh 120 with specimen 138 into cryogenic coolant 152 in cryogenic container 150. Cryogenic coolant 152 can include, for example, liquid nitrogen. Cryogenic coolant 152 may also include slush nitrogen. Other cryogenic coolants such as ethanol, methanol, FC 770 oil (3M) may also be used and all are within the scope of this description.

A variety of rewarming methods can be used to rewarm the cryopreserved biological sample and all are within the scope of this description. In some embodiments, the biological sample may be rewarmed by convective methods and the like.

A variety of biological samples can be cryopreserved according to the systems and methods described herein. In some embodiments, biological samples can be embryos from terrestrial and/or aquatic organisms. In some embodiments, biological samples can be embryos such as Drosophila embryos, mosquito embryos, mouse oocytes, zebrafish embryos, Xenopus laevis oocytes, coral larvae, Lepidochelys olivacea embryos and the like. In some embodiments, the sample can include germplasm—e.g., from a biopsy taken from a testis or an ovary from any animal or species. While described herein in the context of an exemplary embodiment in which the biological samples are Drosophila embryos, the systems and methods described herein can be applied to a variety of biological materials such as, for example, other embryos described herein.

The biological material can be a variably sized biomaterial specimen. The biological material can be any sub-millimeter—or millimeter scale biomaterial. In some embodiments, the term sub-millimeter—or millimeter scale sample can have a largest linear dimension of less than about ten millimeters (mm); or less than about five mm; or less than about one mm; or less than about 0.9 mm; or less than about 0.7 mm; or less than about 0.5 mm; or less than about 0.3 mm; or less than about 0.1 mm; or less than about 50 micrometers; or less than about 10 micrometer; or less than about 1 micrometer.

In some embodiments, the term sub-millimeter—or millimeter scale sample can have a smallest linear dimension of greater than about one micrometer; or greater than about 10 micrometer; or greater than about 0.1 mm; or greater than about 0.3mm; or greater than about 0.5 mm; or greater than about 0.7 mm; or greater than about 0.9 mm; or greater than about one mm; or greater than about five mm; or greater than about ten mm.

Also, while described herein in the context of an exemplary embodiment in which the cryoprotective agent includes ethylene glycol, the composition, systems and methods described herein can involve the use of any suitable cryoprotective agent. Exemplary suitable cryoprotective agents include, but are not limited to, combinations of alcohols, sugars, polymers, and ice blocking molecules that alter the phase diagram of water and allow a glass to be formed more easily (and/or at higher temperatures) while also reducing or controlling the likelihood of ice nucleation and growth during cooling or thawing. In some embodiments, cryopreservative agents may not be used alone, but in combination with other CPA and/or agents that promote cryopreservation. In the case of vitrification solutions, exemplary cryopreservative cocktails are reviewed in Fahy et al., He, Xiaming, et al., Risco, Ramon, et al. and Choi, Jung Kyu, et al. and all incorporated herein by reference. (Fahy et al., Cryobiology 48(1):22-35, 2004; He, Xiaoming, et al. “Vitrification by ultra-fast cooling at a low concentration of cryoprotectants in a quartz micro-capillary: a study using murine embryonic stem cells.” Cryobiology 56.3 (2008): 223-232; Risco, Ramon, et al. “Thermal performance of quartz capillaries for vitrification.” Cryobiology 55.3 (2007): 222-229; Choi, Jung Kyu, Haishui Huang, and Xiaoming He. “Improved low-CPA vitrification of mouse oocytes using quartz microcapillary.” Cryobiology 70.3 (2015): 269-272.) Additional exemplary cryopreservative solutions can include one or more of the following: dimethyl sulfoxide, glycerol, propylene glycol, ethylene glycol, sucrose, trehalose, raffinose, polyvinylpyrrolidone, and/or other polymers (e.g., ice blockers and/or anti-freeze proteins).

In some embodiments, the cryoprotective agent may be present in the composition at various concentrations. In some embodiments, the cryoprotective agent may be present, for example, at a molarity of no more the 6 M such as, for example, no more than 5 M, for example, no more than 4 M, for example, no more than 3 M, for example, no more than 2 M, for example, no more than 1 M, for example, for example, no more than 900 mM, for example, no more than 800 mM, for example, no more than 700 mM, for example, no more than 600 mM, for example, no more than 500 mM, or for example, no more than 250 mM.

In some embodiments, the present description can include methods for cryopreservation of biological specimen, e.g. embryos. In one embodiment, the method can include cryopreservation of Drosophila embryos. In one embodiment, the embryos are Drosophila melanogaster embryos. The cryopreservation of embryos will be described with respect to Drosophila melanogaster embryos but it will be understood that cryopreservation of other embryos are also within the scope of this disclosure.

In some embodiments, the present description can include simple and robust cryopreservation methods for Drosophila embryos such that the embryos can be stored in a cryogenic coolant, e.g. liquid nitrogen, without requiring costly maintenance. Regular Drosophila research labs/centers usually have their own stockroom to maintain the flies. All the flies needs to be transferred to fresh food bottles/vials every 4-6 weeks, which is labor intensive and costly. With the methods described herein, Drosophila embryos can be advantageously stored in liquid nitrogen indefinitely in theory and retrieved for use on demand, lifting enormous financial burden to maintain all the strains. Cryopreservation of Drosophila embryos using the methods described herein can provide enormous advantages including protection against genetic drift, decreased maintenance costs, and reducing the risk of stock loss caused by contamination or accidental mixing of stocks.

In some embodiments, the methods to cryopreserve Drosophila embryos can include embryo collection and staging, embryo dechorionation, embryo permeabilization, cryoprotectant agents (CPA) loading, dehydration and cooling. The method can include rewarming the cryopreserved embryo, CPA unloading and culturing the embryos to form larvae and to adult fruit flies after cryopreservation. In one embodiment, the method for cyropreservation of Drosophila embryos includes the use of the cryomesh described herein.

In some embodiments, methods for cryopreservation of a biological specimen, e.g. Drosophila embryos, can include collection of the embryos and may also include staging of the embryos. The embryos may be collected at any appropriate temperature depending on the temperature suitability for the embryo. In one embodiment, the embryos may be collected at room temperature. The embryos can be placed in a suitable environment to age the embryos to a desired stage for cryopreservation. In one embodiment, the collected embryos can be placed on grape juice plates and incubated at a desired temperature for an incubation duration until the embryos reach a desired embryo stage for cryopreservation. The length of incubation and the incubation temperature can vary and can be adjusted to accommodate the logistics of carrying out the cryopreservation method. The incubation temperature may be increased if it is desired to have a shorter incubation time. Alternatively, the incubation temperature may be decreased if desired, to have a longer incubation time. In one embodiment, the embryos can be incubated between about 18° C. and about 24° C. (Heratherm incubator purchased from Thermo Scientific) for about 15-32 hours. Other incubation temperatures and length of incubation may also be used and all are within the scope of this description. In one embodiment, the embryos can be incubated at about 20° C. for about 22 hours to attain the desired embryo stage.

In some embodiments, the embryos are incubated at one temperature during the staging. In some embodiments, the incubation temperature can be controlled within a narrow window that can result in embryos attaining a desired embryo stage to allow for lower variations of cryopreservation survival rates from batch to batch. In one embodiment, the embryos can be incubated at about 20.1° C. In one embodiment, the incubation temperature can be about 20.1° C. with a tolerance of about +/−0.05° C.

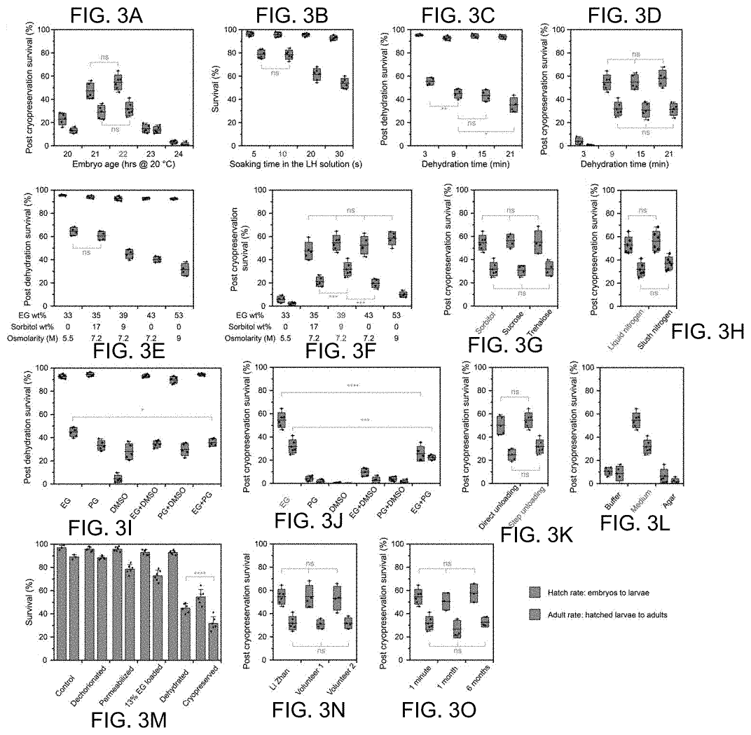

In some embodiments, the gut morphology may be evaluated to verify the embryo stage of the embryos prior to cryopreservation. The embryo stage may be verified under a compound microscope and/or a dissecting microscope. Embryos may be preserved at a variety of stages and cryopreservation with the embryos at any of the stages are within the scope of this description. In some embodiments, the embryos are between about 18 hours and about 24 hours. In some embodiments, embryos of about 22 hours old may be selected and these embryos may have the highest post-cryopreservation survival rate. This can correspond to early stage 16 when head involution and dorsal closure have been completed (FIG. 3A).

In some embodiments, at least some of the Drosophila embryos in a sample to be cryopreserved can be at a stage when head involution and dorsal closure have been completed. In some embodiments, the number of Drosophila embryos in a sample that are at the stage when head involution and dorsal closure have been completed is at least about 10%; or at least about 25%; or at least about 40%; or at least about 50%; or at least about 60%; or at least about 75%; or at least about 90%; or at least about 95%. In some embodiments, all of the Drosophila embryos in a sample to be cryopreserved can be at a stage when head involution and dorsal closure have been completed.

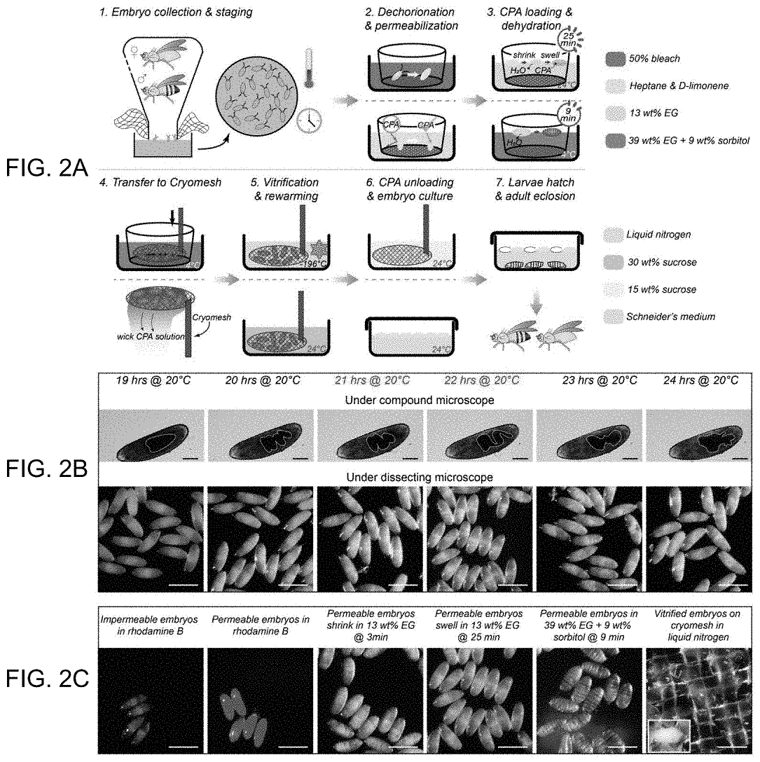

In some embodiments, the method can include correlating the incubation time and temperature with the gut morphology. In some embodiments, gut morpology that can generate the highest cryopreservation rates can be identified and the time and temperature to reach the desired gut morphology can be determined. In some embodiments, the time and temperature that can generate the highest cryopreservation rates can be identified and the gut morphology at the desired time and temperature can be identified. In some embodiments, under the compound microscope, the gut can appear as dark structures (white outlines were manually added to the images for enhanced clarity, FIG. 2B). Under the dissecting microscope, the gut can appear as a milky color (FIG. 2B lower panels). From 19 hrs to 24 hrs, the appearance of the gut can change from a heart-like shaped structure (19 hrs) to a set of 3-4 semi-parallel bars that lie orthogonal to the embryo long axis (20 hrs), that becomes progressively more tilted (21-22 hrs) and can eventually morph into a more extended shape (23-24 hrs). These are approximate incubation times and may vary depending on the exact temperature and strain.

The age of flies used for embryo collection may also impact the cryopreservation survival rates or outcomes. In some embodiments, the age of the flies is between about 1-4 days; or about 5-8 days; or about 9-12 days or greater. In one embodiment, the embryo collection was performed using flies that are about 1-4 days.

In some embodiments, the method can include dechorionating the embryos after incubation to attain the desired stage of the embryos. The dechorionating can include washing the embryos and placing the embryos in a container. In one embodiment, the container can be, for example, a nylon mesh basket. Other containers may be used and all are within the scope of this description. In one embodiment, the dechorionation may be conducted by placing the embryos in a bleach solution for between about two minutes and about four minutes. In some embodiments, the bleach solution may be between about 25% and about 75% bleach. In one embodiment, the dechorionation may be conducted by placing the embryos in about a 50% bleach solution for between about two and about four minutes. After the incubation in the bleach solution, the embryos may be rinsed to remove excess bleach. In one embodiment, the embryos may be rinsed with running tap water for about one to about two minutes to remove excess bleach. The embryos in a container may be briefly blotted on paper towel and placed in a buffer. In one embodiment, the buffer may be a cryobuffer. In one embodiment, the buffer is a isotonic cryobuffer.

In one embodiment, the cryobuffer (20 mM NaCl, 2.7 mM KCl, 10 mM Na₂HPO₄, 1.8 mM KH₂PO₄, 4 mM MgCl₂, 13 mM MgSO₄, 60 mM Glycine, 60 mM Glutamic acid and 5 mM Malic acid, pH6.8, sterilized by filtration) is used. Other cryobuffers may be used and all are within the scope of this description. In one embodiment, embryos may be examined under a dissecting microscope to confirm the removal of chorions.

After dechorionation, the embryos in the mesh basket may be removed from the cryobuffer and blotted on a paper towel to remove as much of the cryobuffer as possible. The embryos in the mesh basket may then be placed in isopropanol for between about five and about 10 seconds. In one embodiment, the embryos in the mesh basket may be dipped in the isopropanol for between about five and about 10 seconds. The mesh basket with embryos inside may be blotted on a paper towel several times to remove the excess isopropanol. The embryos and mesh basket may then be dried by blowing humid air, e.g., using mouth, until the mesh becomes transparent. This drying may be performed to remove any residual isopropanol. Traces of isopropanol, when combined with heptane, may be toxic to the embryos.

In some embodiments, the methods can include permeablizing the embryos. The permeablizing can include placing the embryos in permeablizing solutions. In one embodiment, the permeablizing solutions can include organic solutions. The permeablizing solutions can include, for example, isopropanol, D-Limonene and/or heptane. The length of incubation in the permeabilizing solution, and the permeablizing solutions may vary and all are within the scope of this description.

In one embodiment, the embryos are placed in a container, e.g. mesh basket, and the embryos in the container are transferred into a permeablizing solution. The permeabilization will be described with the use of a mesh basket but it will be understood that other containers may be used to hold the embryos.

In some embodiments, the method can include permeabilizing by transferring the embryos in the mesh basket into a permeabilization solution. In one embodiment, the permeabilization solution is a mixture of D-limonene and heptane (LH). In some embodiments, the permeabilization solution can be a mixture of about 2:1 v/v; or about 3:1 v/v; or about 4:1 v/v; or about 5:1 v/v; or about 6:1 v/v of D-limonene and heptane. In one embodiment, the permeabilization solution is a mixture of about 4:1 v/v of D-limonene and heptane. Other ratios of the D-limonene and heptane may also be used and are within the scope of this description. In some embodiments, the embryos in the mesh basket may be placed in the permeabilizing solution for between about 5 seconds and about 15 second, or for about 10 seconds.

In some embodiments, the embryos and the mesh basket may be removed from the permeabilization mixture and blotted on a paper towed to remove excess liquid. The embryos in the mesh basket may then be placed in heptane for about 5 seconds to remove residual D-limonene around the embryo. The embryos and the mesh basket may then be removed from the heptane and traces of the heptane may be removed by air-drying. The embryos in the mesh basket may then be placed in a buffer such as cryobuffer. In some embodiments, the permeabilization process may take between about 1 and about 2 minutes.

In some embodiments, the method can include loading the embryos with CPA and dehydrating the embryos. In one embodiment, after permeabilizing, a brush may be used to break up clumps into individual embryos floating as a monolayer with minimal overlap. In one embodiment, the mesh basket with the embryos may be blotted and then placed in a CPA loading solution. In some embodiments, the CPA loading solution can include, for example, EG, DMSO, propyleneglycol (PG) and the like in a cryobuffer solution. In one embodiment, the CPA loading solution can be ethylene glycol (EG) in a cryobuffer solution. In some embodiments, the amount of CPA in the CPA loading solution can be between about 10 weight percent and about 20 weight percent. In one embodiment, the CPA loading solution can be about a 13 weight percent EG solution prepared with cryobuffer. Other CPA loading solutions and percentages may also be used and are within the scope of this description.

In one embodiment, the embryos may remain floating in order to maintain access to oxygen when in the CPA solution. The embryos may be in the CPA solution for between about 2 to about 4 minutes; or about 3 minutes. In one embodiment, the embryos may develop “wrinkles” on the embryo surface after about 3 minutes when observed under a dissecting microscope. The “wrinkles” can indicate volumetric shrinkage (i.e., losing water) in response to higher external osmolarity. The percentage of embryos that shrink may be recorded.

In some embodiments, the embryos with the CPA loading solution may be placed in a humid chamber. In some embodiments, the relative humidity may be greater than about 80%. In some embodiments, the “wrinkled” embryos may be placed in the humid chamber until the embryos swell back to their original shape. In one embodiment, the embryos may be placed in the humid chamber from between about 10 minutes to about 45 minutes; or between about 20 minutes to about 30 minutes; or about 25 minutes. In one embodiment, the embryos may be inspected under a dissecting microscope at about 25 minutes to confirm that they swelled back to their original shape. Without being bound by any theory, it is thought that the swelling of the embryos can be indicative of the CPA entry into the embryos. The percentage of embryos that swelled back may be recorded.

In one embodiment, at least about 50% of the embryos may shrink and swell back to their normal size with the CPA loading; or at least about 75%, or at least about 85%; or at least about 90 percent; or at least about 95% may shrink and swell with the CPA. In one embodiment, at least about 90% of the embryos may shrink and swell back to their normal size with the CPA loading.

In some embodiments, the method can include dehydrating the embryos. In some embodiments, the dehydrating may be performed in a dehydrating solution. In some embodiments, the dehydrating solution can include CPAs and a sugar in cryobuffer. In some embodiments, the CPA can include, for example, EG, DMSO, propyleneglycol (PG) and the like. In some embodiments, the sugar in the dehydrating solution can include, for example, sorbitol, sucrose trehalose and the like. In one embodiment, the dehydrating solution can include ethylene glycol and sorbitol in cryobuffer. In some embodiments, the dehydrating solution can include between about 30 weight percent and about 50 weight percent CPA and between about 5 weight percent and about 15 weight percent of sugar in cryobuffer. In one embodiment, the dehydrating solution can include about 39 weight percent EG and about 9 weight percent sorbitol in cryobuffer. Other CPA and sugars may also be used and are within the scope of this description.

In some embodiments, the dehydrating step can include placing the CPA loaded embryos in the dehydrating solution from about 5 minutes to about 15 minutes; or about 9 minutes. In one embodiment, the embryos may be in the dehydrating solution between about 0° C. and about 10° C.; or about 4° C. In some embodiments, the dehydrating of the embryos (i.e., water loss) can elevate the intra-embryonic CPA concentration. This can favor vitrification and avoidance of devitrification during the rewarming processes.

In some embodiments, the dehydrated embryos can be transferred to a cryomesh. In one embodiment, cryomesh can be used to press the floating dehydrated embryos into the dehydrating CPA solution from the top. In one embodiment, nearly all of the embryos can stay attached to the cryomesh when the cryomesh is lifted out of the dehydrating CPA solution. In some embodiments, a wicking agent, e.g. a paper towel, may be used to wick the majority of the remaining dehydrating CPA solution on the cryomesh from the side opposite the embryos. In one embodiment, the wicking process may be performed within 20 seconds since elevated temperature may increase CPA toxicity therefore leading to lower survival. Wicking after about 20 seconds is also within the scope of this description.

In one embodiment, a monolayer of Drosophila embryos can be placed on cryomesh. In one exemplary embodiment, a medium packed monolayer of embryos can occupy about 30% of the total mesh area. In one embodiment, the mesh can be a 20 mm by 20 mm square. Each embryo can occupy 0.07 mm² (=3.14*embryo half length*embryo half width=3.14*0.25 mm*0.09 mm). In one embodiment, a 20 mm*20 mm size mesh can accommodate about 1714 embryos. (=20 mm*20 mm*0.3/0.07) embryos. Meshes of different sizes that can accommodate different numbers of embryos are also within the scope of this description.

In some embodiments, the method can further include cooling for vitrification of the dehydrated embryos. The cryomesh with the dehydrated embryos can be quickly plunged into a cryogenic coolant, e.g. liquid nitrogen. The cryogenic coolant can be liquid nitrogen, slush nitrogen and the like. At this stage the embryos can be cryopreserved and can be stored in the cryogenic coolant until future use.

The vitrified embryos may be stored at cryogenic temperatures for an indefinite period of time and until desired future use. In some embodiments, the embryos may be stored for more than a day; or more than a week; or more than a month; or more than 6 months; or more than a year; or more than 5 years.

In some embodiments, the method can further include rewarming the cryopreserved embryos. A variety of methods can be used to rewarm the embryos and all are within the scope of this description. In one embodiment, the cryopreserved embryos are rewarmed by placing the cryomesh with the vitrified embryos in a rewarming buffer. Rewarming buffers can include buffers with varying amounts of sugars prepared in a buffer, e.g. cryobuffer. In some embodiments, the rewarming buffer may include between about 25 weight percent and about 35 weight percent of a sugar solution in cryobuffer. In one embodiment, the cryomesh with the cryopreserved embryos may be rapidly submerged into a 30 weight percent sucrose solution prepared in the cryobuffer at room temperature while avoiding agitation. Without being bound by any theory, it is thought that the 30 weight percent sucrose in cryobuffer maintains the flattened embryo shape to avoid rapid rehydration and detachment of the embryos from the cryomesh. The cryopreserved embryos may be placed in the rewarming buffer briefly. In some embodiments, the cryopreserved embryos may be placed in the rewarming buffer between about 1 second and about 15 seconds; or between about 3 seconds and about 10 seconds; or about 5 seconds. Incubation times outside of this range are also within the scope of this description.

In some embodiments, the method can further include unloading the CPA from the cryopreserved embryos. In some embodiments, the CPA unloading can be performed by placing the embryos in a CPA unloading buffer. In one embodiment, the CPA unloading buffer can include a solution of a sugar in cryobuffer. In one embodiment, the CPA unloading buffer can include a solution of sucrose in cryobuffer. In some embodiments, the CPA unloading buffer can include between about 5 weight percent and about 25 weight percent; or between about 10 weight percent and about 20 weight percent; or about 15 weight percent of a sugar in a buffer. In one embodiment, the CPA unloading buffer is about a 15 weight percent sucrose in a cryobuffer. Other sugars and cryobuffers may be used and all are within the scope of this description. Concentration of sugars outside of these ranges are also within the scope of this description.

In some embodiments, after a few seconds, e.g., about 5 seconds in the rewarming buffer, e.g. 30 wt % sucrose in cryobuffer, the cryomesh along with the embryos may be transferred to a CPA unloading buffer, e.g. 15 weight percent sucrose prepared in the cryobuffer. In some embodiments, the embryos are placed in the CPA unloading buffer for between about 1 minute and about 10 minutes; or between about 2 minutes and about 5 minutes; or for about 3 min. In one embodiment, the embryos are placed in the CPA unloading buffer for about 3 minutes. In some embodiments, the embryos may be transferred to a cryobuffer to remove all of the intra-embryonic CPA. In some embodiments, the embryos may be placed in the cryobuffer for between about 10 minutes and about 30 minutes; or for about 20 minutes.

In some embodiments, the embryos may be transferred from the cryobuffer into a medium. In one embodiment, the medium is Schneider's medium purchased from Sigma-Aldrich, St. Louis, Mo. Other media that allows culturing of embryos may be used and all are within the scope of this description. In one embodiment, the embryos may be transferred to a 35 mm petri dish filled with 1 ml Schneider medium using a brush. In one embodiment, the embryos may be incubated in a medium overnight. In one embodiment, the embryos may be incubated in a humid chamber overnight.

Incubation of the embryos in the medium overnight can result in formation of larvae, e.g. hatched larvae. In some embodiments, hatched larvae can be transferred, after overnight incubation, from the medium to food vials. Embryo hatch rate can be calculated using the ratio of hatched larvae to total embryos.

The cryopreserved embryos can have a variety of cryopreservation survival rates when rewarmed after cryopreservation. Cryopreservation survival rates can be evaluated by determining the normalized hatch rate, normalized adult rate and/or normalized embryo to adult rate. The normalized survival is the ratio of embryo survival rate for untreated group vs treated group. For example, the survival of embryos without any treatment is 50%, after cryopreservation, the survival of embryos is 20%, then normalized survival is 20%/50%=40%]. Table 3 shows some exemplary cryopreservation survival rates for a 25 different Drosophila strains cryopreserved using the methods described herein.

In some embodiments, the normalized hatch rate can be greater than about 30%; or greater than about 40%; or greater than about 50%; or greater than about 60%; or greater than about 70%; or greater than about 80%; or greater than about 90%.

In some embodiments, the normalized adult rate can be greater than about 10%; or greater than about 20%; or greater than about 30%; or greater than about 40%; or greater than about 50%; or greater than about 60%; or greater than about 70%; or greater than about 80%; or greater than about 90%.

In some embodiments, the normalized embryo to adult rate can be greater than about 10%; or greater than about 20%; or greater than about 30%; or greater than about 40%; or greater than about 50%; or greater than about 60%; or greater than about 70%.

In some embodiments, the food vials with the hatched larvae can be kept at room temperature (i.e., 20-25° C.). In some embodiments, larvae to adult rate can be calculated after 15 days using the ratio of emerged adults to total larvae in the vials. The amount of larvae that are put into the food vial is recorded. The food vial with the larvae is incubated at room temperature. After 15 days, the amount of adult flies in the food vial is recorded. The larvae to adult rate is calculated by the ratio of the quantities of larvae to adult flies.

In some embodiments, the present method can be used to cryopreserve a variety of Drosophila strains. In some embodiments, the Drosophila strains may be wild-type strains. In some embodiments, the Drosophila strains may be strains with one or more mutations.

In some embodiments, the Drosophila strain can be a mutant strain with a mutation. In some embodiments, the mutant strain may be genetically modified to improve the survival rates after cryopreservation. In some embodiments, the mutant strain can be genetically modified while maintaining the original mutation to improve the cryopreservation survival rate. In one embodiment, the genetic modification may be performed by outcrossing with a strain with improved cryopreservation survival rates.

In one embodiment, the method can include collecting embryos from flies that are about 1-4 days and incubating the embryos at about 20° C. for about 22 hours. The method can include soaking the embryos in D-limonene and heptane for about 10 sec for permeabilization. The method can include loading with 13 weight percent EG for 25 min The method can include dehydrating with dehydration solution that can include about 39% EG and 9% sorbitol for a dehydration time of about 9 minutes. The CPA for loading can be EG in cryobuffer. The embryos can be cryopreserved in liquid nitrogen or slush nitrogen. The method can include removing the CPA surrounding the embryos. After removal from cryopreservation, the embryos can be floated on Schneider medium.

### EXAMPLES

### Example 1—Cryopresrvation of Drosophila melanogaster Embryos

Methods

Stock Maintenance

Flies were maintained in Drosophila bottles (6 oz) at room temperature (24.2±0.5° C.). Adults were removed from the bottle after 5-7 days. Fly food was prepared with the same recipe used by the Bloomington Stock Center. (BDSC Cornmeal Food Recipe—Bloomington Drosophila Stock Center)

Cryopreservation Protocol

Step 1. Embryo collection and staging. On day 1, 700-1200 flies at the age of 1-4 days old were used to collect embryos at room temperature. Usually 4 bottles of flies were used, 8 or more bottles were used if needed. Flies were placed in an empty Drosophila bottle covered with a mesh cloth as a cap (FIG. 2A). Embryos were collected in a 1 hour period on a grape juice plate smeared with yeast paste. The first hour collection served as an egg clean-up procedure for the female flies and were abandoned. Disturbance of flies was minimized during embryo collection. Grape juice plates with collected embryos were labeled with the end time point of collection, for instance, 3 pm was used to label the collection from 2 pm to 3 pm. Embryos were placed in a temperature incubator at 20.1±0.05° C. (Heratherm purchased from Thermo Scientific) until reaching the desired stage for cryopreservation. 20° C. was selected so that optimal embryo age for cryopreservation will be achieved during a normal work hour on the following day. In this work, embryo collection occurred in the afternoon and usually 2-4 collections were performed.

To stage the embryos on day 2, for example, the embryo collection labeled as 3 pm on day 1 would reach 22 hrs old at 1 pm on day 2.

Step 2. Dechorionation and permeabilization. On day 2, embryos were washed off from the grape juice plate into a nylon mesh basket and dechorionated in 50% bleach for 2-4 minutes. After rinsing with running tap water for 1-2 minutes to remove excess bleach, embryos along with the mesh basket were briefly blotted on paper towel and placed in the cryobuffer (20 mM NaCl, 2.7 mM KCl, 10 mM Na₂HPO₄, 1.8 mM KH₂PO₄, 4 mM MgCl₂, 13 mM MgSO₄, 60 mM Glycine, 60 mM Glutamic acid and 5 mM Malic acid, pH6.8, sterilized by filtration) in a 35 mm petri dish. Embryos were examined under a dissecting microscope to confirm the removal of chorions. In addition, the gut morphology was evaluated to verify the embryo stage (FIG. 2B).

Before permeabilization, ˜4 ml isopropanol, mixture of D-limonene and heptane (4:1 v/v), and heptane alone were added to three separate 35 mm glass petri dishes in a fume hood. A mesh basket was used to transfer the embryos from one solution to another. Specifically, the mesh basket was lifted from the cryobuffer and blotted on a paper towel to remove as much liquid as possible, followed by a 5-10 second dip in isopropanol until all embryos sank to the bottom. Then, the mesh basket with embryos inside was blotted on a paper towel several times to remove excess isopropanol. The embryos and mesh basket were then dried by blowing humid air (i.e., using mouth) until the mesh became see through (FIG. 26). This step is designed to remove the water on the embryo thereby allowing subsequent exposure to the organic solvent. It is critical to remove the isopropanol by drying since it was noticed that the combination of isopropanol with heptane was toxic to the embryos. Next, the mesh basket was placed in the D-limonene and heptane mixture for 10 seconds to permeabilize the embryo. Similarly, after blotting on a paper towel, the mesh basket was placed in heptane for 5 seconds to remove the D-limonene around the embryo as D-limonene cannot be easily removed by evaporation. Finally, heptane was removed by air drying and the permeabilized embryos along with the mesh basket were placed back into the cryobuffer. The whole permeabilization process usually takes 1-2 min.

Step 3. CPA loading and dehydration. Right after permeabilization, a brush was used to break up clumps into individual embryos floating as a monolayer with minimal overlap (FIG. 2B). The mesh basket was blotted and then placed in 13 weight percent ethylene glycol (EG) solution prepared with cryobuffer in a 35 mm petri dish. The embryos should remain floating in order to maintain access to oxygen. After 3 min, “wrinkles” on the embryo surface were observed under a dissecting microscope, indicating volumetric shrinkage (i.e., losing water) in response to higher external osmolarity (FIG. 2C). The percentage of embryos that shrunk was recorded. The 13 weight percent EG petri dish was then placed in a humid chamber. At 25 min, embryos were inspected under a dissecting microscope to confirm that they swelled back to their original shape, indicating EG had entered the embryos. The percentage of embryos that swelled back was recorded. Usually, if the embryos were at the correct stage and properly permeabilized, >90% embryo would shrink and swell in 13 weight percent EG (FIG. 2C).

Next, the mesh basket was blotted and placed in 39 weight percent EG+9 weight percent sorbitol solution prepared in cryobuffer on ice (i.e., ˜4° C.) for 9 min This step dehydrates the embryos (i.e., water loss) thereby elevating the intra-embryonic EG concentration to favor vitrification and avoidance of devitrification during the rewarming processes. In general, 5-6 ml dehydration CPA was used in a 35 mm petri dish.

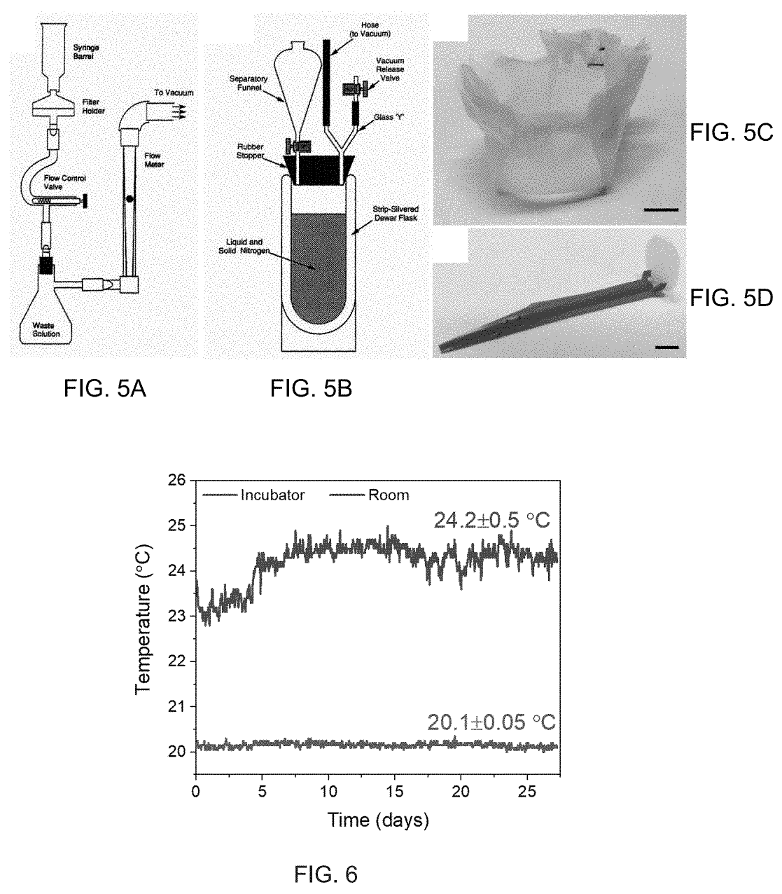

Step 4. Transfer to the cryomesh. After 9 min dehydration, a dry cryomesh was used to press the floating dehydrated embryos into the CPA solution from the top (FIG. 5). Nearly all of the embryos stayed attached to the cryomesh after lifting the cryomesh out of the CPA solution. A paper towel was used to wick the majority of the remaining CPA solution on the cryomesh from the side opposite the embryos. The wicking process should be done within 20 seconds as elevated temperature may increase CPA toxicity therefore leading to lower survival.

Assuming a medium packed monolayer of embryos (i.e., embryos occupy 30% of the total mesh area) and each embryo occupies 0.07 mm² (=3.14*embryo half length*embryo half width=3.14*0.25 mm*0.09 mm), a 2 cm*2 cm size mesh can accommodate 1714 (=20 mm*20 mm*0.3/0.07) embryos.

Step 5. Vitrification and rewarming. The cryomesh with dehydrated embryos was quickly plunged into liquid nitrogen. At this stage the embryos are cryopreserved and can be stored in liquid nitrogen until future use. To rewarm the embryos, the cryomesh was rapidly submerged into 30 weight percent sucrose solution prepared in the cryobuffer (˜40 ml solution in a 50 ml beaker) at room temperature while avoiding agitation. The 30 weight percent sucrose was chosen to maintain the flattened embryo shape to avoid rapid rehydration and detachment of the embryos from the cryomesh.

Step 6. CPA unloading and embryo culture. After a few seconds (i.e., 5 seconds) in 30 weight percent sucrose, the cryomesh along with the embryos were transferred to 15 weight percent sucrose prepared in the cryobuffer for 3 min, followed by transfer to cryobuffer for 20 min to finally remove all of the intra-embryonic CPA. Finally, the embryos were transferred to a 35 mm petri dish filled with 1 ml Schneider medium using a brush. The petri dish was capped and placed in a humid chamber overnight.

Step 7. Larvae hatch and adult eclosion. On day 3, hatched larvae were transferred in the morning from the medium to food vials (15×95 mm shell vial). Embryo hatch rate was calculated using the ratio of hatched larvae to total embryos. The food vials with larvae were kept at room temperature. After 15 days, larvae to adult rate was calculated using the ratio of emerged adults to total larvae in the vials.

Cooling and Warming Rate Measurement

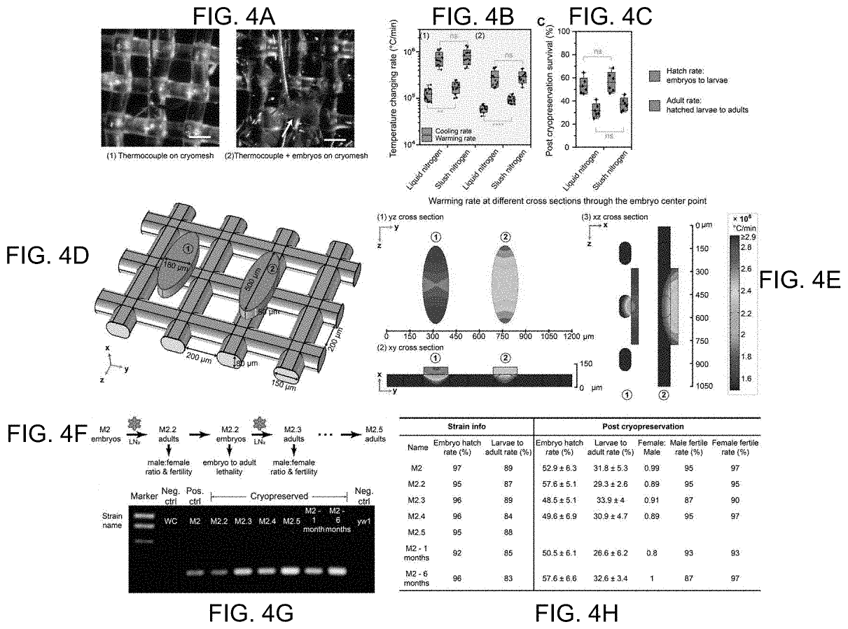

To measure the cooling and warming rates of the cryomesh method, a bare wire type T thermocouple (unsheathed fine gauge thermocouples, wire diameter is 50 μm, OMEGA) and an oscilloscope were used. To test different cryogens, slush nitrogen was prepared by pulling vacuum to cool the liquid nitrogen until slush was formed. The thermocouple was glued to the cryomesh and the temperature was recorded during cooling and warming of the mesh alone. In addition, dehydrated embryos were collected and placed in contact with the thermocouple on the mesh to obtain the corresponding cooling/warming rates for a loaded mesh (FIG. 4). The cooling/warming rates with CPA solutions on the cryomesh were also measured (FIG. 9). Cooling and warming rates were calculated to represent rates during cooling and warming in the temperature zone from −140° C. to −20° C. Importantly, the CPA solutions and CPA loaded Drosophila embryos will be in a glassy phase at −140° C.

Warming Rate Modeling

COMSOL was used to simulate the warming rate of embryos using the cryomesh method. Two extreme conditions were considered: 1) minimal contact between dehydrated embryo and the cryomesh, and 2) maximal contact between the dehydrated embryo and the cryomesh (FIG. 4). The cross section of nylon fibers was set as 150×80 mm, aperture was 200 μm, the length and width of embryo were 500 μm and 180 μm respectively based on direct measurements. To estimate the thickness of dehydrated embryos, the weight of 532 dehydrated embryos was first measured to be 2.6 mg (FIG. 9). The weight of a single dehydrated embryo was then calculated to be 4.9 μg. Assuming the dehydrated embryo density to be the density of embryo solid content (1.37 g/ml) the thickness of dehydrated embryo was estimated to be 50 μm. As the thermal properties of dehydrated embryos are unknown, temperature dependent thermal properties of CPA were used based on previous publications. See Choi et al. Cryobiology 60, 52-70 (2010) and Khosla et al., Langmuir 35, 7364-7375 (2018). For the nylon mesh, the density was set to be 1.15 g/ml, temperature dependent thermal conductivity and heat capacity were obtained from National Institute of Standards and Technology (NIST). Convective heat flux was used as the boundary conditions with convective heat transfer coefficient set as 300 W/(m²*K). Wang et al. CryoLetters 36, 285-288 (2015). Zhang et al. International journal of heat and mass transfer 114, 1-7 (2017). Warming rates at different cross sections through the center point of embryos were compared for two extreme conditions.

In addition, the warming rate of the methods used in previous publications was modeled. See Mazur et al. Science 258, 1932-1935 (1992) and Steponkus et al. Cryo-letters, (1993). Specifically, polycarbonate filter with 10 μm pore size (item #F10013—MB, SPI Supplies) and copper grid for electron microscope with 200 μm aperture (item #G100-Cu, Electron Microscopy Sciences) (See Table 1). Table 1 compares the current methods with previous publications on cryopreservation of Drosophila melanogaster embryos. The CPA solution around the embryos was assumed to be 250 mm thick.

TABLE 1

| | | Mazur et al | Steponkus et al | This work |
|---|---|---|---|---|
| Outcome | Post cryopreservation survival | Hatch rate: 68% | Hatch rate: 83% | Hatch rate^{a}: 88% |
| | using wild type | Adult rate: 40% | Adult rate: 54% | Adult rate: 36% |
| | Multi-generation | x | x | ✓ |
| | cryopreservation? | | | |
| | Long term storage? | x | x | ✓ |
| | Repeated by non-specialist? | x | x | ✓ |
| | Test other mutant strains? | x | x | ✓ |
| | Confirm mutation remained? | x | x | ✓ |
| Key | Embryo staging method | morphology | incubation | morphology + |
| procedure | | | temperature | incubation |
| | | | | temperature |
| | Embryo incubation temperature | Combination of | 25° C. | 20.1 ± 0.05° C. |
| | | 24° C. and 17° C. | | |
| | Permeabilization | Specialized | Poor | Simple device |
| | | device and poor | repeatability | and good |
| | | repeatability | | repeatability |
| | Cryogen used | Slush nitrogen | Slush nitrogen | Liquid nitrogen |
| | Device to hold embryos for | Polycarbonate | EM copper grid | Nylon mesh |
| | cryopreservation | filter | | |
| | CPA solution around embryo | Yes | Yes | Minimal |
| | before vitrification? | | | |
| | Specialized device? | Permeabilization | slush nitrogen | None |
| | Post cryopreservation embryo | setup and slush | maker | Floating on |
| | culture method | nitrogen maker | Immersed in oil | medium |
| | | Placed on agar | | |
| Reference | | 11.15 | 12.13 | |

^{a}WC¹¹¹⁸ was used as the wildtype

Statistics

For plots with two dependent variables, for instance, hatch rate and adult rate, or cooling rate and warming rate, multivariate analysis of variance (MANOVA) and Tukey's post hoc were used for statistical analysis in software SPSS Statistics.

For FIG. 9b, paired two-tailed student's t test were used.

“ns” represents the difference is not statistically significant (p>0.05), *p≤0.05, **p≤0.01, ***p≤0.001, ****p≤0.0001.

Complete statistical analysis including p values for all plots can be find in the separate excel file named Data S1 statistical analysis.

Results

The major challenges to cryopreserve Drosophila melanogaster embryos include embryo age dependent survival, CPA loading, vitrification with scalability, and strain dependent genetic backgrounds. The first hurdle is to introduce CPA directly into the embryo. After dechorionation, the embryos are impermeable to CPA due to the waxy layer and vitelline membrane. Assuming CPA can be loaded, the previous protocols have demonstrated that cryopreservation should be approached through vitrification, a solidification process from liquid into glass with minimal lethal ice formation. Rapid cooling and warming rates are required to achieve cryopreservation via vitrification, even after successful CPA loading. However, it is difficult to scale up the conventional vitrification tools to handle large numbers of Drosophila embryos (i.e., >1000) (See Table 2). Table 2 compares methods utilizing a cryomesh with traditional vitrification tools.

TABLE 2

| | Sample | # of | CPA solution | | |
|---|---|---|---|---|---|
| | volume ^{a} | embryos/ | included in | Cooling rate | Warming rate |
| Device | (μL) | run ^{b} | the sample? | (° C./min) ^{c} | (° C./min) ^{d} |
| Cryotop | 0.1 | 2-3 | yes | ~69,000 | ~117,000 |
| Copper grid | ~1 | ~25 | yes | ~24,000 | ~25,000 |
| Open pulled straw | ~2 | ~50 | yes | ~15,000 | ~40,000 |
| Quartz capillary | ~2 | ~50 | yes | ~30,000 | ~30,000 |
| Traditional straw ^{e} | 500 | ~12,500 | yes | ~1,384 | ~896 |
| Cryomesh | NA ^{f} | >1,700 | no | ~59,600 | ~280,000 |

^{a} This volume includes CPA solution and biomaterials to be cryopreserved unless otherwise noted.
^{b} Estimated value based on previous publication. ~25 Drosophila embryos per microliter was reported in previous publication using the copper grid.(41)
^{c} Cooling by plunging into liquid nitrogen.
^{d} Warming by convective method.
^{e} Devitrification occurs during rewarming, leading to low warming rate.
^{f} This is the actual volume of cryopreserve biomaterials themselves, for example, the volume of one dehydrated embryo is estimated to be 3.6 nL (see calculation under “Warming rate modeling” in the Supplementary materials). More than 1700 Drosophila embryos can be placed on one cryomesh (2 cm * 2 cm) in a monolayer.

The protocol above was successfully validated with 25 Drosophila strains from different sources. Importantly, the protocol showed significant improvement over previous published efforts supporting wide adoption by the Drosophila community.

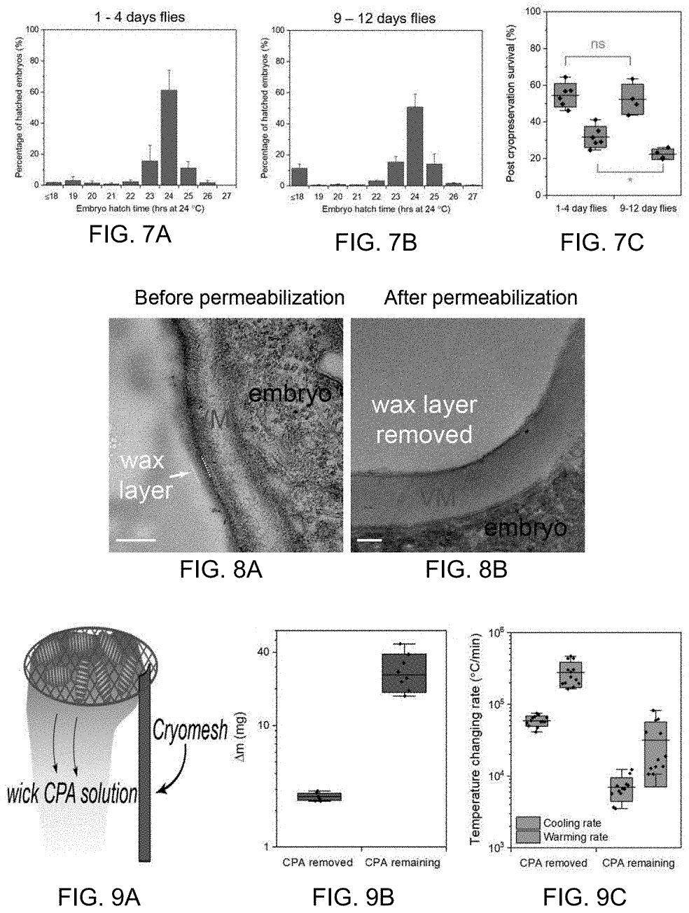

Extensive optimization was performed on each step of the protocol using a stock strain named M2 (FIG. 2A). As a derivative of WC¹¹¹⁸, M2 carries a traceable single nucleotide polymorphisms (SNP) on the X chromosome and is homozygous, viable and fertile. Embryo survival was evaluated by hatch rate (embryo to larvae) and adult rate (hatched larvae to adult). While embryo age was reported to significantly affect cryopreservation outcomes in previous studies, little guidance was provided to identify and reproducibly obtain the optimal age for non-specialists. In addition, the embryonic development rate is highly temperature dependent. A robust procedure was established to stage the embryos by combining chronological age via strict control of incubation time at a set incubation temperature (i.e., 20.1±0.05° C., FIG. 6), and morphological features via inspection of embryo gut appearance under the compound and/or dissecting microscopes (FIG. 2B). Specifically, under the compound microscope, the gut appeared as dark structures (white outlines were manually added to the images for enhanced clarity, FIG. 2B). Under the dissecting microscope, the gut appeared a milky color (FIG. 2B lower panels). From 19 hrs to 24 hrs, the appearance of the gut changed from a heart-like shaped structure (19 hrs) to a set of 3-4 semi-parallel bars that lie orthogonal to the embryo long axis (20 hrs), that become progressively more tilted (21-22 hrs) and eventually morph into a more extended shape (23-24 hrs). By cryopreserving embryos at various age, it was established that 22 hrs old embryos provided the highest post cryopreservation survival, which corresponds to early stage 16 when head involution and dorsal closure have been completed (FIG. 3A). For embryos at older ages, the impermeable cuticle layer starts to form, precluding the uptake of CPA and therefore survival decreased sharply. The age of flies used for embryo collection also impacted the cryopreservation outcome. A lower adult rate was observed using older flies (9-12 days) than young ones (1-4 days), potentially due to female egg retention which led to lower embryonic stage uniformity (FIG. 7).

As a critical step, a simple mesh basket was employed, in contrast to the specialized device in the prior art, to perform permeabilization using the mixture of D-limonene and heptane (LH) (FIG. 5). We found that 10 second soaking time in the LH solution was adequate for permeabilization and caused minimal injury (FIG. 3B). Permeable embryos stained red in rhodamine B solution and showed removal of the wax layer when visualized by electron microscope (FIG. 2C, FIG. 8). In general, embryo permeable CPAs include ethylene glycol (EG), propylene glycol (PG) and dimethyl sulfoxide (DMSO), while sugars such as sucrose, sortibol and trehalose are non-permeable. To introduce CPA into the embryos for subsequent vitrification, a monolayer of embryos were initially exposed to low concentration permeable CPA (i.e., 13 weight percent). More than 90% of the embryos first lost water and shrank due to higher external osmolarity, followed by swelling as CPA slowly enters until reaching equilibrium (FIG. 2C).

At this point, intra-embryonic CPA concentration was elevated through dehydration by placing the embryos in a high concentration CPA (i.e., ˜39 weight percent) at 4° C. Dehydrated embryos appeared flat in shape with multiple “wrinkles” on the surface (FIG. 2C). The final intra-embryonic CPA concentration is a function of dehydration time, total osmolarity and permeable CPA concentration of the dehydration CPA. Higher intra-embryonic CPA concentration results in greater the protection against lethal ice formation during ensuing cooling and rewarming, but also greater toxicity. To achieve the optimal balance, a number of parameters were compared—the post dehydration survival (i.e., CPA toxicity) and post cryopreservation survival using different dehydration time, dehydration CPA concentrations and dehydration CPA compositions. Under the same weight concentration, EG has proven to have the least CPA toxicity and highest survival post cryopreservation (FIG. 3E-F). The neurotoxicity of DMSO has been reported, which may contribute to DMSO having the highest CPA toxicity shown in FIG. 3I. In addition, the use of permeable CPA cocktails did not outperform individual permeable CPAs. However, a combination of permeable and non-permeable CPAs reduces CPA toxicity and provides superior post cryopreservation survival, compared to permeable CPAs alone with the same total osmolarity (FIG. 3E-F). Further, when 39 weight percent EG+9 weight percent sorbitol was used as the dehydration CPA, post cryopreservation survival remained similar with increasing dehydration time from 9 min to 21 min. Replacing sorbitol with sucrose or trehalose did not affect post cryopreservation survival (FIG. 3G). To reduce the cost of the reagents and minimize the time of the protocol, 9 min dehydration in 39 weight percent EG+9 weight percent sorbitol was selected.

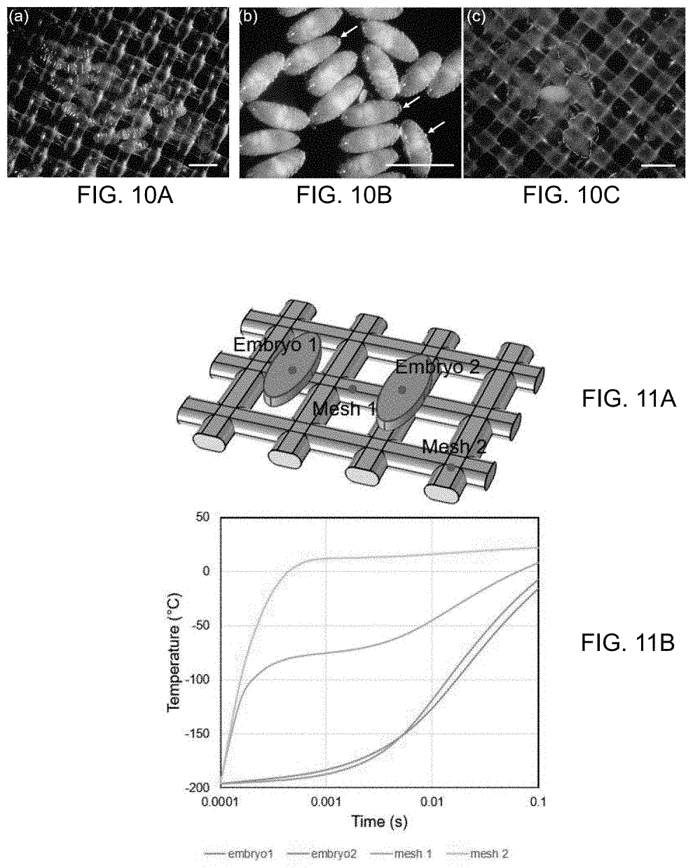

To cryopreserve embryos in large quantities, the cryomesh was used—a nylon mesh attached to a thin polystyrene holder. A 2 cm by 2 cm size mesh can easily accommodate ˜1700 embryos. Almost all of the embryos were transferred to the cryomesh within seconds by pressing a dry cryomesh into the dehydration CPA solution and lifting it out (FIG. 2A). Importantly, it was demonstrated that prior to vitrification, wicking the remaining CPA solution off the cryomesh, significantly improved the cooling and warming rates, as well as the post cryopreservation survival (FIG. 3H, FIG. 10). This “excess CPA solution free” method maximizes the cooling and warming rate while allowing the processing of large numbers of embryos thereby outperforming traditional vitrification tools (Table 2). The cryomesh with the embryos was then quickly plunged into liquid nitrogen (LN₂) for vitrification and can be stored in LN₂ until future use. Vitrified embryos appeared transparent in LN₂ while crystallized embryos (i.e., failure) looked white (FIG. 2C, FIG. 10).

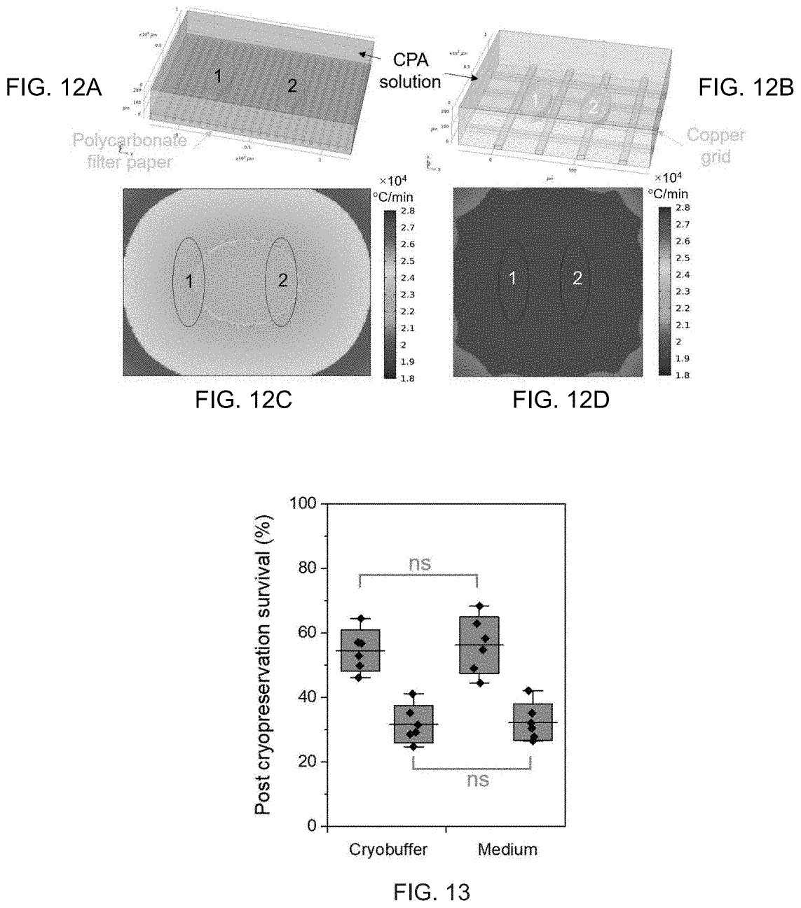

Slush nitrogen (SN₂) was also tested. A thermocouple was placed in contact with the embryos and recorded a faster cooling rate in SN₂ but similar warming rate compared with LN₂ (FIG. 4A-B). Further, similar post cryopreservation survival was shown between LN₂ and SN₂ therefore LN₂ was selected due to the easier accessibility (FIG. 4C). Heat transfer simulation suggested that the larger the contact area of the embryo with the cryomesh, the faster they rewarmed as the nylon mesh rewarmed faster than the embryos (FIG. 4D-E, FIG. 11). Modeling implied the average warming rates of embryos with minimum and maximum mesh contact was 2.2×10⁵ ° C./min, consistent with the experimental measurement (FIG. 4B). In addition, modeling indicated similar warming rates throughout each embryo (FIG. 4E). This characterization suggests a dramatically higher warming rates over previous publication where embryos were surrounded by CPA solution (i.e., ˜2×10⁴ ° C./min, Table 2, FIG. 12). This is a critical protocol improvement as recent studies suggested that high rewarming rate is the vital step in vitrification based cryopreservation and can even “rescue” poorly cooled biomaterials with certain amount of ice present.

For intra-embryonic CPA removal after rewarming, dehydrated embryos were exposed to 15 weight percent sucrose solution prior to the cryobuffer (i.e., a isotonic saline buffer) to mitigate the osmotic shock. Direct unloading in the cryobuffer was also tested, which surprisingly showed a similar hatch rate but slightly lower adult rate (FIG. 3K). This likely indicates that the vitelline membrane helped to avoid overswelling of the dehydrated embryos (FIG. 10b). Further, cost of cryopreservation was demonstrated to be greatly reduced by using a cryobuffer as the carrier solution to prepare CPA and unloading solutions, supported by the equivalent post cryopreservation survival compared with Schneider medium (FIG. 13). Different embryo culture methods were tested as they are now permeable and vulnerable to external environment (FIG. 3L). Floating on Schneider medium provided the best survival compared to floating on the cryobuffer and placed on agar. Indeed, Schneider medium supplied essential nutrients for further development and an aqueous environment for continuous unloading of intra-embryonic CPA. Using the optimal cryopreservation protocol, stepwise survival of strain M2 is presented in FIG. 3M. After cryopreservation, the hatch rate and adult rate were 52.9±6.3% and 31.8±5.3%, compared to 97% and 89% for untreated embryos.

Next, the ease of application and robustness of the protocol was tested by training two non-specialist volunteers (notably including one high school student) and post cryopreservation characterization of M2. Both volunteers obtained consistent post cryopreservation survival (FIG. 3N). This demonstrates the simplicity and translatability of the developed protocol. Additional storage time in liquid nitrogen including 1 month and 6 months was carried out. The adults that survived from cryopreserved embryos of M2 were named to be M2.2. To investigate the impact of repeated cryopreservation cycles, the embryos from the adults that survived the cryopreservation were collected and cryopreserved, and repeated for multiple generations (i.e., M2.2-M2.5, FIG. 4F). In FIG. 4H, all the progenies showed similar embryo to adult survival compared to M2. Equal sex ratio suggests that no lethal mutations were introduced on the X chromosome after repeated cryopreservations or long term liquid nitrogen storage. In addition, comparable post cryopreservation survival and fertility were retained across multiple generations and different liquid nitrogen storage time. Importantly, we demonstrated that original SNP in the M2 strain was maintained after cryopreservation using PCR (FIG. 4H).

Finally, the protocol was validated with 24 other strains. Wildtype, mutant, single balancer and double balancers were covered from different sources including the Bloomington Stock Center, our lab and other Drosophila labs (Table 3). Table 3 shows normalized post cryopreservation survival of 25 different Drosophila strains using the same protocol.

TABLE 3

| | | | | Post cryopreservation | | |
|---|---|---|---|---|---|---|
| Strain info | | | | | | Normalized |
| Strain | | Hatch | Adult | Normalized | Normalized | embryo to |
| name | Genetics | rate (%) | rate (%) | hatch rate (%) | adult rate (%) | adult rate (%) |
| WC3b | Single 3^{rd} from WC | 56 | 58 | 83.6 ± 15.4 | 64.4 ± 16.1 | 51.8 ± 9.3 |
| OR | Oregon-R | 91 | 77 | 68.6 ± 5.1 | 73.2 ± 8.9 | 50.1 ± 6.1 |
| WC1b | Single X from WC | 84 | 74 | 71.3 ± 8.9 | 53 ± 5 | 41.3 ± 8.7 |
| GFP ^{a} | yw; Dfd-GFP | 62 | 81 | 74.3 ± 14.8 | 53.3 ± 14.1 | 40.4 ± 15.2 |
| WC1 | Another single X from WC | 94 | 89 | 76.9 ± 8.8 | 44.4 ± 7.2 | 33.9 ± 5.7 |
| WC | WC-1118 | 96 | 83 | 88 ± 4 | 36.4 ± 6 | 32 ± 5.5 |
| M2-3b | Single 3^{rd} from M2 | 81 | 73 | 58 ± 6.7 | 50.4 ± 7.5 | 29.2 ± 6 |
| WC3 | Another single 3^{rd} from WC | 93 | 89 | 72.2 ± 8.4 | 35 ± 9.5 | 26 ± 9.9 |
| S3 | Dhc6-12, FRT/TM3 | 46 | 61 | 53.6 ± 8 | 46.3 ± 11.4 | 24.4 ± 5.4 |
| S11 ^{b} | Actβ80/UAC-D-GFP | 56 | 58 | 67.1 ± 5.7 | 34.6 ± 6.6 | 23.6 ± 6.2 |
| NS1 | yw; Sp/CyO; | 49 | 57 | 48.9 ± 3.6 | 49.4 ± 12.4 | 22.2 ± 4.9 |
| | (X from strain GFP) | | | | | |
| WC1.1 | X from WC1 | 95 | 60 | 52.3 ± 2.5 | 38.8 ± 4.4 | 20.3 ± 2.1 |
| yw1 | yw chromosome strain GFP | 89 | 90 | 51.2 ± 5.6 | 40.1 ± 9.4 | 20.1 ± 2.8 |
| M2 | single nucleotide | 97 | 89 | 54.5 ± 6.5 | 35.7 ± 5.9 | 19.7 ± 5.2 |
| | polymorphisms (SNP) on X | | | | | |
| S4 | w; Sp/CyO; TM2/TM6 | 21 | 44 | 52.2 ± 6.6 | 35 ± 4.6 | 18.5 ± 4.6 |
| S7 | DhcGFP11-3/TM3 Sb | 64 | 61 | 54.1 ± 3.2 | 32.9 ± 3.5 | 17.5 ± 2.3 |
| S8 | X; TM3 Sb/TM6B Tb | 36 | 74 | 52.1 ± 9.3 | 26.8 ± 10.8 | 14.5 ± 8.4 |
| S5 | elav-ANFGFP; TM3/TM6 | 56 | 61 | 40.5 ± 5.1 | 33.3 ± 2.6 | 13.6 ± 2.8 |
| S6 | Sp-EM6/FM7-GFP | 74 | 84 | 75.8 ± 7.1 | 17.2 ± 2.4 | 12.9 ± 1.3 |
| S10 | w; B1/CyO; TM2/TM6 | 22 | 44 | 48.6 ± 6.5 | 26.2 ± 11.8 | 12 ± 3.8 |
| S2 | po ros/w, FM6 | 67 | 51 | 51.2 ± 5.6 | 22.8 ± 7.5 | 11.9 ± 4.9 |
| WC2 | Single 2^{nd} from WC | 91 | 85 | 62.1 ± 10.1 | 17.3 ± 4 | 10.6 ± 2.7 |
| S9 | w; B1/CyO; TM2/TM6, | 24 | 54 | 45.1 ± 8 | 20.6 ± 3.8 | 9.6 ± 3.5 |
| | UAS-GAL80 | | | | | |
| S12 | po ros/w, FM6; Sp/CyO | 50 | 44 | 41.4 ± 5.4 | 22 ± 7 | 9.1 ± 3.3 |
| S1 | w; Sp/CyO | 68 | 50 | 36.2 ± 7.1 | 8.7 ± 4.5 | 3 ± 1.5 |

* normalized survival = survival post cryopreservation/survival without any treatment.
^{a} Obtained from Bloomington Drosophila Stock Center, stock number is 30877
^{b} Obtained from Dr. Michael O'Connor's lab

To investigate whether the optimized conditions for M2 shown in FIG. 3 applies for other strains, each variable was tested with at least two other strains (Table 4). Table 4 shows an overview of optimized variables in the cryopreservation procedures.

TABLE 4

| Variable name | Tested conditions* |
|---|---|
| Age of flies used for embryo collection | 1-4 days; 9-12 days |
| Embryo stage/age in 20° C. incubator | 20 hrs; 21 hrs; 22 hrs; |
| | 23 hrs; 24 hrs |
| Soaking time in D-limonene & heptane | 5 s; 10 s; 20 s; 30 s |
| solution for permeabilization | |
| Dehydration CPA concentration | 33% EG; 43% EG; |
| | 39% EG + 9% sorbitol; |
| | 35% EG + 17% sorbitol; |
| | 53% EG. All units are weight percent |
| Dehydration time | 3 min; 9 min; 15 min; 21 min |
| CPA and/or cocktails | EG; PG; DMSO; EG + PG; EG + DMSO; |
| | PG + DMSO |
| Carrier solution to prepare CPA | Cryobuffer; Schneider medium |
| and unloading solution | |
| Cryogen | Liquid nitrogen; slush nitrogen |
| Removing CPA around embryos on | Yes; no |
| cryomesh before cooling? | |
| CPA unloading method | Direct unloading; step unloading |
| Post cryopreservation embryo | Float on Schneider medium; |
| culture method | float on cryobuffer; placed on agar |

*Optimal conditions are underlined

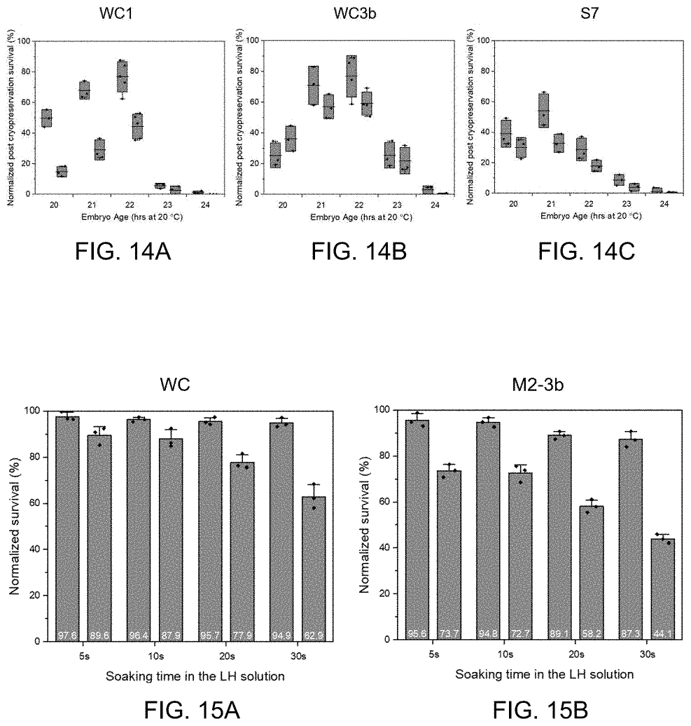

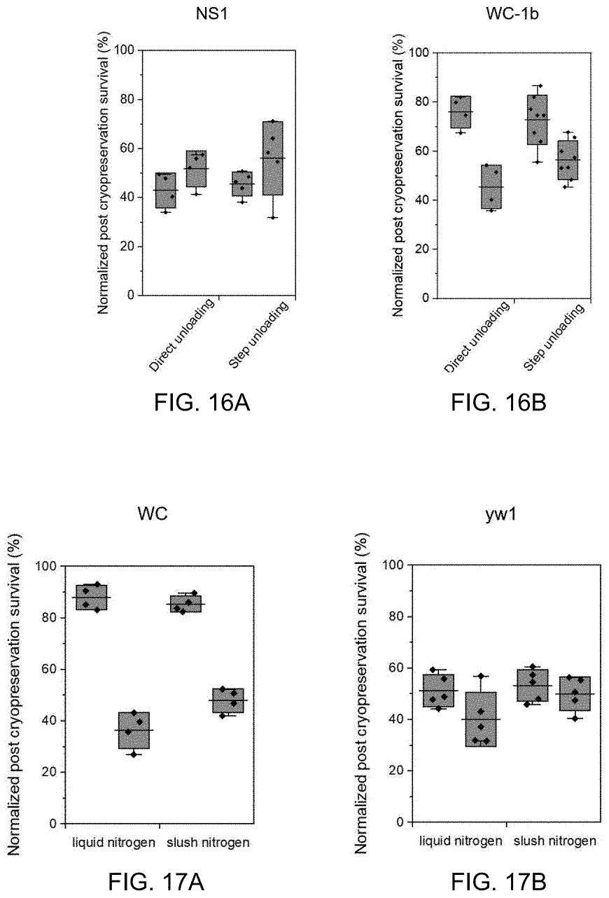

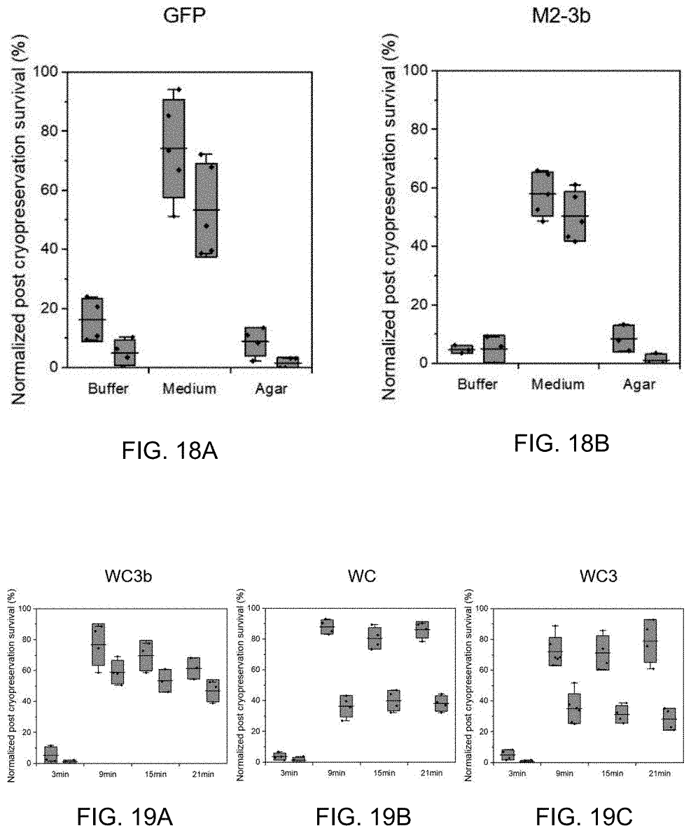

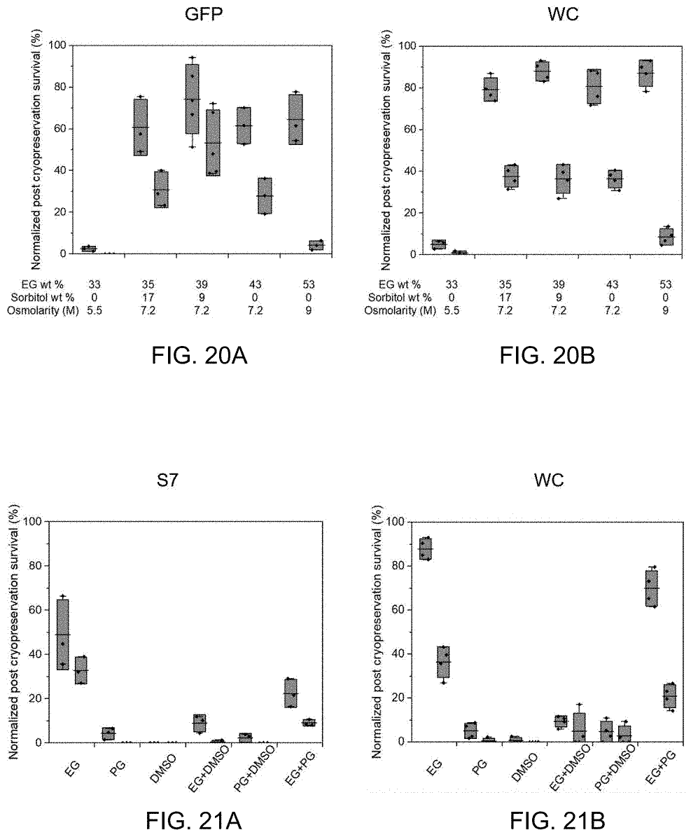

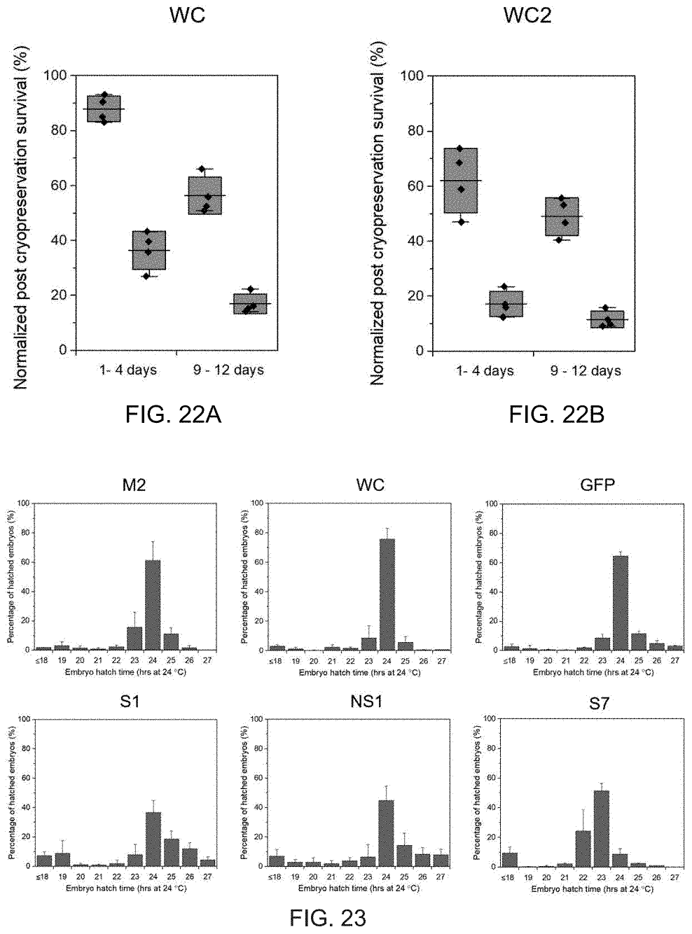

The same optimal conditions were shown, except for the variable embryo age, apply across strains (FIG. 14-22). Specifically, for strain S7, 21 hrs old embryos provided higher post cryopreservation survival than 22 hrs old embryos due to slightly faster embryonic developmental rate or increased egg retention time (FIG. 23). In addition, as genetic crosses are routinely performed in Drosophila labs, new strains were derived by crossing them to explore the impact on cryopreservation outcome. For example, WC1b was generated by crossing a single WC¹¹¹⁸ male to S2 strain to isogenize the X chromosome. Table 1 showed the summary of the post cryopreservation survival normalized by embryos without any treatment. Comparable survival post cryopreservation with previous publications was achieved using wildtype (WC¹¹¹⁸,).

Although strain dependent survival was noted, higher than 10% normalized embryo to adult rate can be achieved in the majority of strains (Table 1). A second chromosome balancer stock S1 yielded very low embryo to adult survival. To investigate whether the genetic background variations of S1 caused this low survival rate, S1 to the GFP strain that exhibits a higher survival rate post cryopreservation was outcrossed. The resultant strain, NS1, retained its second chromosome balancer, yet showed improved post cryopreservation survival (Table 3), demonstrating that survival rates can be improved by outcrossing to mitigate genetic background contributions that impact cryopreservation.

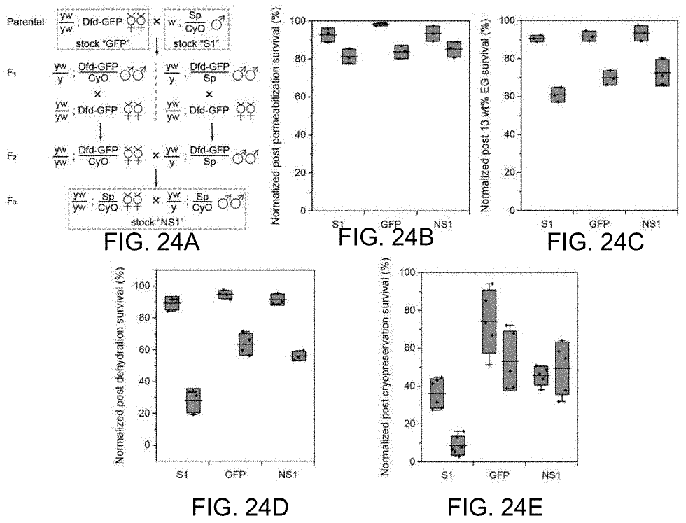

To explore factors underlying the strain dependent survival following cryopreservation, the contribution of embryo age distribution was examined. One hour embryo collections from different strains were incubated at 24° C. and the hatch frequency at various times was recorded (FIG. 23). It was observed that strains M2, WC, and GFP showed a narrow embryo age distribution while strains S1, NS1 and S7 have a broader distribution. In fact, various egg retention patterns regulated by genetics have been reported (28, 29). As post cryopreservation survival depends on embryo age upon vitrification, strains with modest egg retention (i.e., narrow embryo age distribution) could potentially have higher post cryopreservation survival rates. Beside genetic variation, it was shown that a clutch of embryos from older parent flies display a broader range of ages, than did embryos collected from younger parent flies (FIG. 7). In the case of S1 and NS1, post cryopreservation survival still varied despite similar broad embryo age distribution (FIG. 23). Analysis of the stepwise survival during the cryopreservation procedure indicates that the genetic variation between S1 and NS1 result in discrepant tolerance to CPA toxicity (FIG. 24).

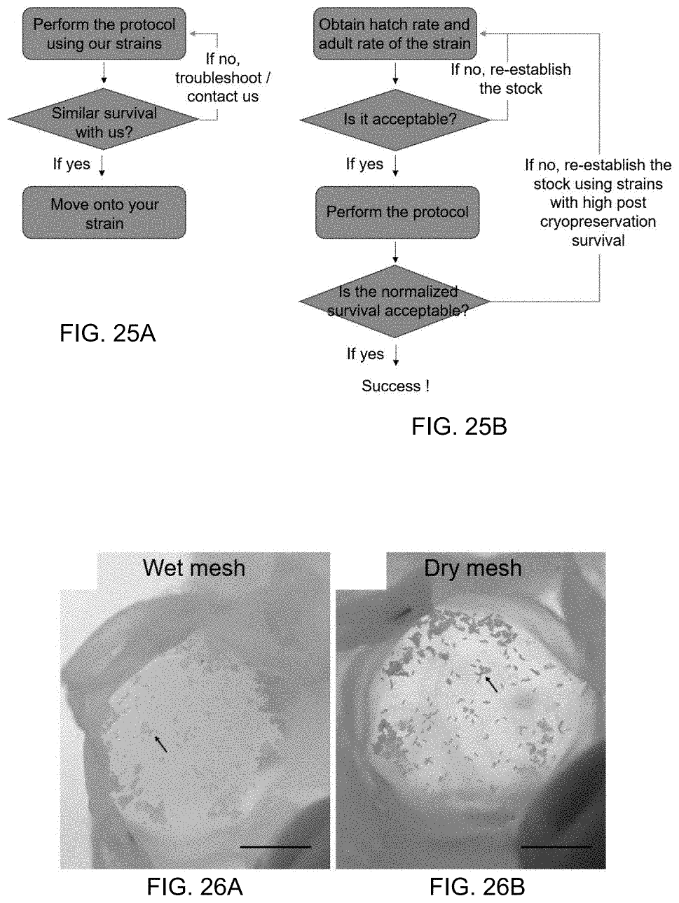

To adopt the protocol for any new lab strain, the flowchart shown in FIG. 25 can be followed using one of the high survival strains reported here as a positive control. Cryopreservation of Drosophila stocks will significantly reduce the cost of stock maintenance and stabilize the genotypes, facilitating genetic and evolutionary studies by halting introduction of mutations and genetic drift in stocks.

All ranges given are intended to further include “any range therebetween” whether or not this is affirmatively stated.

All publications, patents and patent documents are incorporated by reference herein, as though individually incorporated by reference, each in their entirety, as though individually incorporated by reference. In the case of any inconsistencies, the present disclosure, including any definitions therein, will prevail.

Although specific embodiments have been illustrated and described herein, any arrangement that achieve the same purpose, structure, or function may be substituted for the specific embodiments shown. This application is intended to cover any adaptations or variations of the example embodiments of the invention described herein. These and other embodiments are within the scope of the following claims and their equivalents.

What is claimed is:

1. A method for cryopreservation of Drosophila embryos comprising:
collecting Drosophila embryos;
treating embryos for cryopreservation, wherein the treating comprises staging the embryos, dechorionating the embryos, permeabilizing the embryos, loading the embryos with a cryoprotective solution and dehydrating the loaded embryos, the cryoprotective solution comprising a cryoprotective agent (CPA);
transferring the embryos to a cryomesh and removing excess cryoprotective solution; and
cooling the embryos by placing the embryos on the cryomesh in a cryogenic coolant for cryopreservation of the Drosophila embryos.

2. The method of claim 1, wherein the staging of the embryos comprises visually evaluating the gut morphology of the embryo.

3. The method of claim 1, wherein the staging of the embryos comprises incubating the embryos until the embryos are at a stage when head involution and dorsal closure has been completed.

4. The method of claim 1, wherein the staging of the embryos comprises incubating the embryos in an incubator at about 20° C. for about 22 hours.

5. The method of claim 1, wherein the dechorionating comprises incubating the embryos in about 50 weight percent bleach.

6. The method of claim 1, wherein the permeabilizing comprises incubating in a permeabilization solution comprising D-limonene and heptane.

7. The method of claim 1, wherein the cryoprotective solution comprises ethylene glycol (EG), propylene glycol (PG), dimethyl sulfoxide (DMSO) and combinations thereof.

8. The method of claim 1, wherein the dehydrating comprises incubation in a dehydrating solution, wherein the dehydrating solution comprises the CPA and a sugar.

9. The method of claim 1, wherein the removing excess cryoprotective solution comprises wicking the cryomesh with the embryos to remove liquid surrounding the embryos prior to placement in the cryogenic coolant.

10. The method of claim 1, further comprising rewarming the embryos after cryopreservation.

11. The method of claim 10, wherein the rewarming comprises rewarming in a rewarming buffer, unloading the CPA from the cryopreserved embryos and culturing the embryos in a medium, wherein the rewarming buffer comprises sucrose, trehalose and combinations thereof.

12. The method of claim 11, wherein the culturing comprises culturing the embryos in Schneider's medium for between about 8 hours and about 24 hours to form larvae.

13. The method of claim 1, wherein the Drosophila comprises a wild-type strain or a mutant strain.

14. The method of claim 1, wherein the Drosophila comprises a mutant strain with a mutation and wherein the mutant strain is genetically modified while maintaining the mutation to improve the survival rates after cryopreservation.

15. A method for maintaining stocks of Drosophila strains comprising:
collecting Drosophila embryos;
treating embryos for cryopreservation, wherein the treating comprises staging the embryos, dechorionating the embryos, permeabilizing the embryos, loading the embryos with a cryoprotective solution and dehydrating the cryoprotective solution loaded embryos;
transferring the embryos to a cryomesh and removing excess cryoprotective solution; and
cooling the embryos by placing the embryos on the cryomesh in a cryogenic coolant for cryopreservation of the Drosophila embryos; and
rewarming the embryos after cryopreservation and culturing the rewarmed embryos in medium.

16. The method of claim 15, wherein the method minimizes the genetic drift in stocks.

17. The method of claim 15, wherein the method halts introduction of further mutations due to genetic drift.

18. A method for cryopreservation of embryos comprising:
collecting the embryos;
treating embryos for cryopreservation, wherein the treating comprises staging the embryos, dechorionating the embryos, permeabilizing the embryos, loading the embryos with a cryoprotective solution and dehydrating the cryoprotective solution loaded embryos, wherein the cryoprotective solution comprises a cryoprotective agent (CPA);
transferring the embryos to a cryomesh and removing excess cryoprotective solution; and
cooling the embryos by placing the embryos on the cryomesh in a cryogenic coolant for cryopreservation of the embryos.

19. The method of claim 18, wherein the embryos are terrestrial organism embryos and/or aquatic organism embryos.

20. The method of claim 18, wherein the embryos are Drosophila embryos.
